(19) United States
(12) Patent Application Publication
(10) Pub. No.: US 2021/0346324 A1
(43) Pub. Date: Nov. 11, 2021

(54) Niclosamide Formulations and Methods of Use

(71) Applicant: NeuroBo Therapeutics, Inc., Boston, MA (US)

(72) Inventors: Akash BAKSHI, Foster City, CA (US); Nadja MANNOWETZ, Foster City, CA (US); Andrew BARTYNSKI, Foster City, CA (US)

(21) Appl. No.: 17/308,712

(22) Filed: May 5, 2021

Related U.S. Application Data
(60) Provisional application No. 63/020,520, filed on May 5, 2020.
(60) Provisional application No. 63/049,084, filed on Jul. 7, 2020.
(60) Provisional application No. 63/078,864, filed on Sep. 15, 2020.
(60) Provisional application No. 63/113,763, filed on Nov. 13, 2020.

(51) Int. Cl. A61K 31/167; A61K 9/48; A61P 31/14

(52) U.S. Cl. CPC A61K 31/167; A61K 9/4858; A61K 9/16; A61K 9/4866; A61P 31/14; A61K 9/485

(57) ABSTRACT

Disclosed are niclosamide formulations for use in the treatment and prevention of viral infection. In some embodiments, the infection is transduced by a coronavirus, including a SARS-Cov-2 virus.

### CROSS-REFERENCE TO RELATED APPLICATIONS

This application claims priority to U.S. Provisional Application No. 63/020,520, filed May 5, 2020; U.S. Provisional Application No. 63/049,084, filed Jul. 7, 2020; U.S. Provisional Application No. 63/078,854, filed Sep. 15, 2020; and U.S. Provisional Application No. 63/113,763, filed Nov. 13, 2020, all of which are incorporated herein by reference in their entireties.

### FIELD OF THE DISCLOSURE

The present invention is directed to formulations of niclosamide or pharmaceutically acceptable salt thereof for use in dry solid formulations and methods of using the same, particularly in methods of treating and preventing viral infection.

### BACKGROUND

A long-felt and unmet need in the art exists for an efficacious universal anti-viral agent that can treat or prevent viral transmission or infection. The disclosure prevents formulations and methods to solve this need.

### SUMMARY

The disclosure provides a formulation comprising an amount of niclosamide, wherein the niclosamide comprises a dry and solid form, and one or more of one or more of a surfactant, an excipient, a pH modulator, a preservative, a binder, a glidant, a dispersant, a disintegrant and a filler. In some embodiments, the glidant or the dispersant comprises talc. In some embodiments, the formulation comprises niclosamide and one or more of a glidant and a dispersant. In some embodiments, the formulation comprises niclosamide and one or more of a glidant and a dispersant, wherein the glidant or the dispersant comprises talc. In some embodiments, the formulation comprises niclosamide and one or more of a glidant and a dispersant in a ratio of 2:1. In some embodiments, the formulation comprises niclosamide and one or more of a glidant and a dispersant in a ratio of 2:1, wherein the glidant or the dispersant comprises talc.

In some embodiments of the formulations of the disclosure, the formulation comprises a plurality of nanospheres, a plurality of microspheres, a plurality of particulates, a powder, a pill, a capsule, or a tablet. In some embodiments, the niclosamide comprises a plurality of nanospheres, a plurality of microspheres, a plurality of particulates, a powder, a pill, a capsule, or a tablet. In some embodiments, the one or more of the surfactant, the excipient, the pH modulator, the preservative, the binder, the glidant, the dispersant, the disintegrant and the filler comprises a plurality of nanospheres, a plurality of microspheres, a plurality of particulates, a powder, a pill, a capsule, or a tablet.

In some embodiments of the formulations of the disclosure, the formulation comprises one or more compartment(s). In some embodiments, the one or more compartment(s) comprise at least two compartments and wherein the at least two compartments are configured to comprise at least one inner compartment and at least one outer compartment. In some embodiments, the at least one inner compartment or the at least one outer compartment comprises a controlled-release formulation. In some embodiments, the at least one inner compartment or the at least one outer compartment comprises a sustained-release formulation. In some embodiments, the at least one inner compartment or the at least one outer compartment comprises an immediate-release formulation.

In some embodiments of the formulations of the disclosure, the excipient or the surfactant increases the permeability of a tissue within the subject to the niclosamide. In some embodiments, the tissue comprises an endothelial membrane. In some embodiments, a component of the intestinal tract or the stomach comprises the tissue. In some embodiments, a component of the respiratory tract comprises the tissue. In some embodiments, a component of the blood brain barrier comprises the tissue.

In some embodiments of the formulations of the disclosure, one or more of the surfactant, the excipient, the pH modulator, the preservative, the binder, the glidant, the dispersant, the disintegrant and the filler increase the in vivo stability of the formulation or of the niclosamide.

In some embodiments of the formulations of the disclosure, one or more of the surfactant, the excipient, the pH modulator, the preservative, the binder, the glidant, the dispersant, the disintegrant and the filler increase the shelf life or in vitro stability of the formulation or of the niclosamide.

In some embodiments of the formulations of the disclosure, one or more of the surfactant, the excipient, the pH modulator, the preservative, the binder, the glidant, the dispersant, the disintegrant and the filler increase the biodistribution of the formulation or of the niclosamide.

In some embodiments of the formulations of the disclosure, one or more of the surfactant, the excipient, the pH modulator, the preservative, the binder, the glidant, the dispersant, the disintegrant and the filler maintain a constant circulating blood plasma concentration of the formulation or of the niclosamide.

In some embodiments of the formulations of the disclosure, a liquid or a suspension comprises the formulation or the niclosamide. In some embodiments, the formulation is provided in a unit dosage form. In some embodiments, the unit dosage form comprises a single dose of the formulation and wherein a single dose comprises an amount of niclosamide effective to reduce, partially or completely, a risk of infection by a virus. In some embodiments, the unit dosage form comprises at least two doses of the formulation and wherein a single dose comprises an amount of niclosamide effective to reduce, partially or completely, a risk of infection by a virus.

In some embodiments of the formulations of the disclosure, a food or a beverage comprises the formulation. In some embodiments, the combination of the food or the beverage and the formulation forms a suspension. In some embodiments, the combination of the food or the beverage and the formulation forms a liquid. In some embodiments, the formulation dissolves in the food or the beverage. In some embodiments, the food or the beverage comprises one or more of a dietary supplement, a meal replacement, an electrolyte, a protein, a sugar and a pharmaceutical carrier. In some embodiments, the food or the beverage comprises a pharmaceutical carrier and wherein one or more of an intravenous tube, a feeding tube or a central line comprise the formulation. In some embodiments, the pharmaceutical carrier or the formulation comprises one or more of a nanoparticle or a polymer. In some embodiments, the food or the beverage comprises a pediatric formula, a diabetic formula or a senior formula. In some embodiments, the food or the beverage comprises caffeine. In some embodiments, the food or the beverage comprises alcohol.

In some embodiments of the formulations of the disclosure, the formulation is suitable for systemic administration. In some embodiments, the systemic administration comprises administration via an oral, inhaled, or intravenous route.

In some embodiments of the formulations of the disclosure, including those in which the formulation is suitable for systemic administration, the formulation comprises a dry powder and wherein the formulation is nebulized, aerosolized, or vaporized for inhaled systemic administration. In some embodiments, the niclosamide formulation is suitable for use or provided for use in a nebulizer as a nebulized formulation. In some embodiments, the niclosamide formulation is suitable for use or provided for use in a humidifier or inhaler as a droplet or aerosolized formulation. In some embodiments, the niclosamide formulation is suitable for use or provided for use in a humidifier or inhaler as a droplet or an aerosolized formulation. In some embodiments, the niclosamide formulation is suitable for use or provided for use in a vaporizer a vaporized formulation. In some embodiments, the vaporized formulation is suitable for use or provided for use in a vaporizing device, including, but not limited to a vaporizer or vapor pen (also referred to as a vape pen).

In some embodiments of the formulations of the disclosure, including those in which the formulation is suitable for systemic administration, the formulation comprises a dry powder and wherein the formulation is solubilized for injected systemic administration.

In some embodiments of the formulations of the disclosure, the niclosamide comprises at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 80%, at least 95%, at least 97%, at least 99% or at least any percentage in between weight of niclosamide by weight of the formulation. In some embodiments, the niclosamide comprises about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 80%, about 95%, about 97%, about 99% or about any percentage in between weight of niclosamide by weight of the formulation. In some embodiments, the niclosamide comprises 1%, 5%, 10%, 15%, 20%, 25%, 30%, 35%, 40%, 45%, 50%, 55%, 60%, 65%, 70%, 75%, 80%, 85%, 80%, 95%, 97%, 99% or any percentage in between weight of niclosamide by weight of the formulation.

In some embodiments of the formulations of the disclosure, the formulation comprises niclosamide comprises between 0.1 and 5 grams of niclosamide. In some embodiments, the formulation comprises niclosamide comprises 500 milligrams (mg) of niclosamide. In some embodiments, the formulation comprises niclosamide comprises 1 gram of niclosamide. In some embodiments, the formulation comprises niclosamide comprises 2 grams of niclosamide.

In some embodiments of the formulations of the disclosure, the formulation comprises one or more of mannitol, microcrystalline cellulose (MCC), sodium lauryl sulfate (SLS), Sodium Starch Glycolate, Hydroxypropyl cellulose (HPC), Colloidal Silicon Dioxide, and Sodium Stearyl Fumarate. In some embodiments, the niclosamide comprises between 50% and 60% weight by weight of the formulation. In some embodiments, the niclosamide comprises between 54% and 57% weight by weight of the formulation. In some embodiments, the niclosamide comprises between 55% and 56% weight by weight of the formulation. In some embodiments, the niclosamide comprises 55% weight by weight of the formulation. In some embodiments, the niclosamide comprises 55.6% weight by weight of the formulation. In some embodiments, the formulation comprises mannitol and wherein the mannitol comprises between 4% and 12% weight by weight of the formulation. In some embodiments, the formulation comprises mannitol and wherein the mannitol comprises between 6% and 10% weight by weight of the formulation. In some embodiments, the formulation comprises mannitol and wherein the mannitol comprises 8% weight by weight of the formulation. In some embodiments, the formulation comprises mannitol and wherein the mannitol comprises 8.8% weight by weight of the formulation. In some embodiments, the formulation comprises MCC and wherein the MCC comprises between 12% and 18% weight by weight of the formulation. In some embodiments, the formulation comprises MCC and wherein the MCC comprises between 14% and 16% weight by weight of the formulation. In some embodiments, the formulation comprises MCC and wherein the MCC comprises 15% weight by weight of the formulation. In some embodiments, the formulation comprises MCC and wherein the MCC comprises 15.1% weight by weight of the formulation. In some embodiments, the formulation comprises SLS and wherein the SLS comprises at least 3% weight by weight of the formulation. In some embodiments, the formulation comprises SLS and wherein the SLS comprises between 3% and 7% weight by weight of the formulation. In some embodiments, the formulation comprises SLS and wherein the SLS comprises between 4% and 6% weight by weight of the formulation. In some embodiments, the formulation comprises SLS and wherein the SLS comprises 5% weight by weight of the formulation. In some embodiments, the formulation comprises Sodium Starch Glycolate and wherein the Sodium Starch Glycolate comprises between 4% and 12% weight by weight of the formulation. In some embodiments, the formulation comprises Sodium Starch Glycolate and wherein the Sodium Starch Glycolate comprises between 7% and 9% weight by weight of the formulation. In some embodiments, the formulation comprises Sodium Starch Glycolate and wherein the Sodium Starch Glycolate comprises 8% weight by weight of the formulation. In some embodiments, the formulation comprises HPC and wherein the HPC comprises between 3% and 7% weight by weight of the formulation. In some embodiments, the formulation comprises HPC and wherein the HPC comprises between 4% and 6% weight by weight of the formulation. In some embodiments, the formulation comprises HPC and wherein the HPC comprises 5% weight by weight of the formulation. In some embodiments, the formulation comprises Colloidal Silicon Dioxide and wherein the Colloidal Silicon Dioxide comprises between 0.1% and 0.9% weight by weight of the formulation. In some embodiments, the formulation comprises Colloidal Silicon Dioxide and wherein the Colloidal Silicon Dioxide comprises between 0.4% and 0.6% weight by weight of the formulation. In some embodiments, the formulation comprises Colloidal Silicon Dioxide and wherein the Colloidal Silicon Dioxide comprises 0.5% weight by weight of the formulation. In some embodiments, the formulation comprises Sodium Stearyl Fumarate and wherein the Sodium Stearyl Fumarate comprises between 0.1% and 4% weight by weight of the formulation. In some embodiments, the formulation comprises Sodium Stearyl Fumarate and wherein the Sodium Stearyl Fumarate comprises between 1% and 3% weight by weight of the formulation. In some embodiments, the formulation comprises Sodium Stearyl Fumarate and wherein the Sodium Stearyl Fumarate comprises 2% weight by weight of the formulation.

The term “about” as used throughout the disclosure encompasses an increase or a decrease from the recited value up to 10%, inclusive of the endpoints. For example, “about 50%” as used throughout the disclosure includes a range from 45% to 55% as well as 50%. In some embodiments, the deviation from the recited value does not qualitatively change a recited property of the recited value or the recited component of the formulation.

In some embodiments of the formulations of the disclosure, the formulation comprises one or more of mannitol, microcrystalline cellulose (MCC), sodium lauryl sulfate (SLS), Sodium Starch Glycolate, Hydroxypropyl cellulose (HPC), Colloidal Silicon Dioxide, and Sodium Stearyl Fumarate. In some embodiments, the niclosamide comprises between about 50% and about 60% weight by weight of the formulation. In some embodiments, the niclosamide comprises between about 54% and about 57% weight by weight of the formulation. In some embodiments, the niclosamide comprises between about 55% and about 56% weight by weight of the formulation. In some embodiments, the niclosamide comprises about 55% weight by weight of the formulation. In some embodiments, the niclosamide comprises about 55.6% weight by weight of the formulation. In some embodiments, the formulation comprises mannitol and wherein the mannitol comprises between about 4% and about 12% weight by weight of the formulation. In some embodiments, the formulation comprises mannitol and wherein the mannitol comprises between about 6% and about 10% weight by weight of the formulation. In some embodiments, the formulation comprises mannitol and wherein the mannitol comprises about 8% weight by weight of the formulation. In some embodiments, the formulation comprises mannitol and wherein the mannitol comprises about 8.8% weight by weight of the formulation. In some embodiments, the formulation comprises MCC and wherein the MCC comprises between about 12% and about 18% weight by weight of the formulation. In some embodiments, the formulation comprises MCC and wherein the MCC comprises between about 14% and about 16% weight by weight of the formulation. In some embodiments, the formulation comprises MCC and wherein the MCC comprises about 15% weight by weight of the formulation. In some embodiments, the formulation comprises MCC and wherein the MCC comprises about 15.1% weight by weight of the formulation. In some embodiments, the formulation comprises SLS and wherein the SLS comprises at least about 3% weight by weight of the formulation. In some embodiments, the formulation comprises SLS and wherein the SLS comprises between about 3% and about 7% weight by weight of the formulation. In some embodiments, the formulation comprises SLS and wherein the SLS comprises between about 4% and about 6% weight by weight of the formulation. In some embodiments, the formulation comprises SLS and wherein the SLS comprises about 5% weight by weight of the formulation. In some embodiments, the formulation comprises Sodium Starch Glycolate and wherein the Sodium Starch Glycolate comprises between about 4% and about 12% weight by weight of the formulation. In some embodiments, the formulation comprises Sodium Starch Glycolate and wherein the Sodium Starch Glycolate comprises between about 7% and about 9% weight by weight of the formulation. In some embodiments, the formulation comprises Sodium Starch Glycolate and wherein the Sodium Starch Glycolate comprises about 8% weight by weight of the formulation. In some embodiments, the formulation comprises HPC and wherein the HPC comprises between about 3% and about 7% weight by weight of the formulation. In some embodiments, the formulation comprises HPC and wherein the HPC comprises between about 4% and about 6% weight by weight of the formulation. In some embodiments, the formulation comprises HPC and wherein the HPC comprises about 5% weight by weight of the formulation. In some embodiments, the formulation comprises Colloidal Silicon Dioxide and wherein the Colloidal Silicon Dioxide comprises between about 0.1% and about 0.9% weight by weight of the formulation. In some embodiments, the formulation comprises Colloidal Silicon Dioxide and wherein the Colloidal Silicon Dioxide comprises between about 0.4% and about 0.6% weight by weight of the formulation. In some embodiments, the formulation comprises Colloidal Silicon Dioxide and wherein the Colloidal Silicon Dioxide comprises about 0.5% weight by weight of the formulation. In some embodiments, the formulation comprises Sodium Stearyl Fumarate and wherein the Sodium Stearyl Fumarate comprises between about 0.1% and about 4% weight by weight of the formulation. In some embodiments, the formulation comprises Sodium Stearyl Fumarate and wherein the Sodium Stearyl Fumarate comprises between about 1% and about 3% weight by weight of the formulation. In some embodiments, the formulation comprises Sodium Stearyl Fumarate and wherein the Sodium Stearyl Fumarate comprises about 2% weight by weight of the formulation.

The disclosure provides a formulation comprising (a) niclosamide, comprising 55% weight by weight of the formulation; (b) mannitol, comprising 8% weight by weight of the formulation; (c) MCC, comprising 15% weight by weight of the formulation; (d) SLS, comprising 5% weight by weight of the formulation; (e) Sodium Starch Glycolate, comprising 8% weight by weight of the formulation; (f) Hydroxypropyl cellulose (HPC), comprising 5% weight by weight of the formulation; (g) Colloidal Silicon Dioxide, comprising 0.5% weight by weight of the formulation; and (h) Sodium Stearyl Fumarate, comprising 2% weight by weight of the formulation.

The disclosure provides a formulation comprising (a) niclosamide, comprising about 55% to about 56% weight by weight of the formulation; (b) mannitol, comprising about 8% to about 9% weight by weight of the formulation; (c) MCC, comprising about 15% to about 16% weight by weight of the formulation; (d) SLS, comprising about 5% weight by weight of the formulation; (e) Sodium Starch Glycolate, comprising about 8% weight by weight of the formulation; (f) Hydroxypropyl cellulose (HPC), comprising about 5% weight by weight of the formulation; (g) Colloidal Silicon Dioxide, comprising about 0.5% weight by weight of the formulation; and (h) Sodium Stearyl Fumarate, comprising about 2% weight by weight of the formulation.

The disclosure provides a formulation comprising (a) 250 mg of niclosamide; (b) 39.6 mg of mannitol, (c) 68.1 mg of MCC; (d) 22.5 mg of SLS; (e) 36 mg of Sodium Starch Glycolate; (f) 22.5 mg of Hydroxypropyl cellulose (HPC); (g) 2.3 mg of Colloidal Silicon Dioxide; and (h) 9 mg of Sodium Stearyl Fumarate.

The disclosure provides a formulation comprising (a) about 250 mg of niclosamide; (b) about 39.6 mg of mannitol, (c) about 68.1 mg of MCC; (d) about 22.5 mg of SLS; (e) about 36 mg of Sodium Starch Glycolate; (f) about 22.5 mg of Hydroxypropyl cellulose (HPC); (g) about 2.3 mg of Colloidal Silicon Dioxide; and (h) about 9 mg of Sodium Stearyl Fumarate.

The disclosure provides a formulation comprising (i) intra-granular components and (ii) extra-granular components; wherein the intra-granular components comprise: (a) niclosamide, comprising about 55% to about 56% weight by weight of the formulation; (b) mannitol, comprising about 8% to about 9% weight by weight of the formulation; (c) MCC, comprising about 15% to about 16% weight by weight of the formulation; (d) SLS, comprising about 5% weight by weight of the formulation; (e) Sodium Starch Glycolate, comprising about 5% weight by weight of the formulation; (f) Hydroxypropyl cellulose (HPC), comprising about 5% weight by weight of the formulation; (g) Colloidal Silicon Dioxide, comprising about 0.3% weight by weight of the formulation; and (h) Sodium Stearyl Fumarate, comprising about 1% weight by weight of the formulation; and wherein the extra-granular components comprise: (i) Sodium Starch Glycolate, comprising about 3% weight by weight of the formulation; (j) Colloidal Silicon Dioxide, comprising about 0.2% weight by weight of the formulation; and (k) Sodium Stearyl Fumarate, comprising about 1% weight by weight of the formulation.

The disclosure provides a formulation comprising (i) intra-granular components and (ii) extra-granular components; wherein the intra-granular components comprise: (a) niclosamide, comprising about 55.6% weight by weight of the formulation; (b) mannitol, comprising about 8.8% weight by weight of the formulation; (c) MCC, comprising about 15.1% weight by weight of the formulation; (d) SLS, comprising about 5% weight by weight of the formulation; (e) Sodium Starch Glycolate, comprising about 5% weight by weight of the formulation; (f) Hydroxypropyl cellulose (HPC), comprising about 5% weight by weight of the formulation; (g) Colloidal Silicon Dioxide, comprising about 0.3% weight by weight of the formulation; and (h) Sodium Stearyl Fumarate, comprising about 1% weight by weight of the formulation; and wherein the extra-granular components comprise: (i) Sodium Starch Glycolate, comprising about 3% weight by weight of the formulation; (j) Colloidal Silicon Dioxide, comprising about 0.2% weight by weight of the formulation; and (k) Sodium Stearyl Fumarate, comprising about 1% weight by weight of the formulation.

The disclosure provides a formulation comprising (i) intra-granular components and (ii) extra-granular components; wherein the intra-granular components comprise: (a) about 250 mg of niclosamide; (b) about 39.6 mg of mannitol; (c) about 68.1 mg of MCC; (d) about 22.5 mg of SLS; (e) about 22.5 mg of Sodium Starch Glycolate; (f) about 22.5 mg of Hydroxypropyl cellulose (HPC); (g) about 1.4 mg of Colloidal Silicon Dioxide; and (h) about 4.5 mg of Sodium Stearyl Fumarate; and wherein the extra-granular components comprise: (i) about 13.5 mg of Sodium Starch Glycolate; (j) about 0.9 mg of Colloidal Silicon Dioxide; and (k) about 4.5. mg of Sodium Stearyl Fumarate.

In some embodiments of the formulations of the disclosure, the formulation is in a capsule.

In some embodiments of the formulations of the disclosure, the formulation is not a tablet.

In some embodiments of the formulations of the disclosure, the formulation shows a higher percentage of drug release than a reference formulation comprising 500 mg niclosamide, maize starch, talcum, sodium lauryl sulphate, povidone, vanillin, magnesium stearate, and saccharin sodium. In some embodiments, the formulation provides a higher percentage of drug release than a percentage of drug release of a reference formulation. In some embodiments, the reference formulation comprises 500 mg niclosamide, maize starch, talcum, sodium lauryl sulphate, povidone, vanillin, magnesium stearate, and saccharin sodium. In some embodiments, the reference formulation comprises a tablet comprising 500 mg niclosamide, maize starch, talcum, sodium lauryl sulphate, povidone, vanillin, magnesium stearate, and saccharin sodium.

In some embodiments of the formulation of the disclosure, the formulation provides a Cₘₐₓ after oral dosing that is between about 1.5 and 2.5 times higher than a reference formulation comprising niclosamide, maize starch, talcum, sodium lauryl sulphate, povidone, vanillin, magnesium stearate, and saccharin sodium. In some embodiments, the reference formulation is YOMESAN.

In some embodiments of the formulations of the disclosure, the formulation shows higher percentage of drug release than a reference formulation comprising niclosamide and an amount of SLS that is less than 2.5% weight by weight of the formulation. In some embodiments, the formulation provides a higher percentage of drug release than a percentage of drug release from a reference formulation. In some embodiments, the reference formulation comprises 500 mg niclosamide, maize starch, talcum, sodium lauryl sulphate, povidone, vanillin, magnesium stearate, and saccharin sodium. In some embodiments, the reference formulation comprises a tablet comprising 500 mg niclosamide, maize starch, talcum, sodium lauryl sulphate, povidone, vanillin, magnesium stearate, and saccharin sodium.

In some embodiments of the formulations of the disclosure, the formulation has systemic bioavailability higher than about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% when administered orally. In some embodiments, the formulation provides a systemic bioavailability of equal to or greater than 10%, 15%, 20%, 25%, 30%, 35%, 40%, 45%, 50%, 55%, 60%, 65%, 70%, 75%, 80%, 85%, 90%, 95%, 99% or any percentage in between. In some embodiments, the formulation has higher systemic bioavailability than a reference formulation comprising 500 mg niclosamide, maize starch, talcum, sodium lauryl sulphate, povidone, vanillin, magnesium stearate, and saccharin sodium. In some embodiments, the formulation has a higher systemic bioavailability than the systemic bioavailability of a reference formulation. In some embodiments, the reference formulation comprises 500 mg niclosamide, maize starch, talcum, sodium lauryl sulphate, povidone, vanillin, magnesium stearate, and saccharin sodium. In some embodiments, the reference formulation comprises a tablet comprising 500 mg niclosamide, maize starch, talcum, sodium lauryl sulphate, povidone, vanillin, magnesium stearate, and saccharin sodium.

In some embodiments of the formulations of the disclosure, including those formulations comprising one or more of mannitol, microcrystalline cellulose (MCC), sodium lauryl sulfate (SLS), Sodium Starch Glycolate, Hydroxypropyl cellulose (HPC), Colloidal Silicon Dioxide, and Sodium Stearyl Fumarate, the formulation comprises a plurality of spheres. In some embodiments, the formulation comprises a plurality of microspheres. In some embodiments, the formulation comprises a dry or solid particulate, optionally, compressed into a plurality of spheres or microspheres. In some embodiments, the formulation comprises a dry or solid particulate, optionally, compressed into a plurality of spheres or microspheres, wherein the plurality of spheres or microspheres are enclosed or encapsulated. In some embodiments, the formulation comprises a capsule comprising a dry or solid particulate, optionally, compressed into a plurality of spheres or microspheres. In some embodiments, the capsule is administered orally. In some embodiments, the capsule is broken, opened or separated to release the dry or solid particulate, optionally, compressed into a plurality of spheres or microspheres into a liquid to generate a liquid or a suspended formulation. In some embodiments, the liquid or the suspended formulation is administered orally (oral route), intravenously (intravenous route), intrajejunally (intrajejunal route), intrathecally (intrathecal route), intraspinally (intraspinal route). In some embodiments, the liquid or the suspended formulation is dispersed as wet formulation. In some embodiments, the liquid or the suspended formulation is atomized, vaporized, nebulized, aerosolized, or otherwise dispersed as a wet formulation for inhaled or intranasal administration.

The disclosure provides a formulation of the disclosure, for use in treating or preventing an infection transduced by a virus, comprising administering an effective amount of the formulation to the subject, wherein, upon contacting an infectious agent, the formulation prevents transmission, infection, replication, survival or growth of the virus in the subject.

In some embodiments of the uses of the formulations of the disclosure, contacting an infectious agent comprises the subject contacting directly or indirectly an organism infected with the virus or contaminated by the virus. In some embodiments, the organism presents one or more sign(s) or symptom(s) of an infection. In some embodiments, the organism does not present a sign or a symptom of an infection. In some embodiments, the organism is an asymptomatic carrier of the virus.

In some embodiments of the uses of the formulations of the disclosure, contacting comprises communication of the virus through air or through fluid media. In some embodiments, the fluid comprises a bodily fluid or particulate thereof from the infected or contaminated organism. In some embodiments, the bodily fluid particulate comprises an exhaled or an expelled droplet. In some embodiments, the bodily fluid particulate comprises an aerosolized droplet. In some embodiments, the bodily fluid comprises sputum, saliva, blood, plasma, serum, lymph fluid, tears, sweat, urine or feces.

In some embodiments of the uses of the formulations of the disclosure, the virus is communicated to the subject from across a physical distance of between 0.1 and 12 feet from the organism. In some embodiments, the virus is communicated to the subject from across a physical distance of 6 feet or less from the organism.

In some embodiments of the uses of the formulations of the disclosure, the virus survives, remains viable or retains an infectious activity on a surface for between 0.1 minute and 6 months. In some embodiments, the surface comprises a biological surface. In some embodiments, the surface comprises one or more of a plant, a tree, a crop, skin, hair, nails or a component thereof. In some embodiments, the surface comprises one or more of silk, cotton, cellulose, cork, wool, wood, cardboard, latex, rubber and paper.

In some embodiments of the uses of the formulations of the disclosure, the virus survives, remains viable or retains an infectious activity on a surface for between 0.1 minute and 6 months. In some embodiments, the surface does not comprise a biological surface.

In some embodiments of the uses of the formulations of the disclosure, the virus survives, remains viable or retains an infectious activity on a surface for between 0.1 minute and 6 months. In some embodiments, the surface comprises an organic surface. In some embodiments, the surface comprises one or more of carbon fiber, a plastic and synthetic fiber.

In some embodiments of the uses of the formulations of the disclosure, the virus survives, remains viable or retains an infectious activity on a surface for between 0.1 minute and 6 months. In some embodiments, the surface comprises an organic surface. In some embodiments, the surface comprises an inorganic surface. In some embodiments, the surface comprises one or more of a metal, silicone and glass.

In some embodiments of the uses of the formulations of the disclosure, the organism infected with the virus or contaminated with the virus is a human.

In some embodiments of the uses of the formulations of the disclosure, the organism infected with the virus or contaminated with the virus is not a human. In some embodiments, the organism is a mammalian organism, an avian organism, a reptilian organism, an amphibian organism, a crustacean organism, an arthropod organism or a chordata organism.

In some embodiments of the uses of the formulations of the disclosure, the virus is a zoonotic virus.

In some embodiments of the uses of the formulations of the disclosure, contacting comprises consumption or handling of the organism by the subject. In some embodiments, the organism is a domesticated animal. In some embodiments, the domesticated animal is a pet or an ornamental animal. In some embodiments, the pet or ornamental animal is a dog, a cat, a bird, a reptile or a rodent. In some embodiments, the rodent is a mouse, a rat, a rabbit, a hare or a hamster.

In some embodiments of the uses of the formulations of the disclosure, contacting comprises consumption or handling of the organism by the subject. In some embodiments, the organism is a domesticated animal. In some embodiments, the organism is a live-stock animal.

In some embodiments of the uses of the formulations of the disclosure, contacting comprises consumption or handling of the organism by the subject. In some embodiments, the organism is a wild animal.

In some embodiments of the uses of the formulations of the disclosure, contacting occurs within a distance of 5 miles or less of: i) a human dwelling, ii) a laboratory or a research facility, iii) a market, store, or retail location, iv) a zoo, game reserve, wildlife reserve, land managed for wildlife protection or wildlife sanctuary, v) a farm, a field, or an agricultural location, vi) a hotel, a lodge, a resort or a site for an ecotourism activity, vii) a source of water, a well or a barrel maintained for drinking water, a stream, a river, a lake and an ocean, and/or viii) an airplane, a ship, a boat, a bus, a train, a car or a truck. In some embodiments, the organism is maintained in one or more of i) through viii).

In some embodiments of the uses of the formulations of the disclosure, contacting the organism is intentional. In some embodiments, the organism is personal property, state property, communal property, a hunting target, a food source, a research subject, a pet, a native species, an invasive species, a prey of any one of the foregoing or a predator of any one of the foregoing.

In some embodiments of the uses of the formulations of the disclosure, contacting the organism is unintentional. In some embodiments, the organism is a native species, an invasive species or a predator of the subject.

In some embodiments of the uses of the formulations of the disclosure, the virus has a mutation rate of between 10⁻⁸ and 10⁻⁴ mutations per nucleotide of the genome per replication cycle.

In some embodiments of the uses of the formulations of the disclosure, the virus has a mutation rate of between 10⁻⁵ and 10⁻² nucleotide substitutions per site per year. In some embodiments, the virus has a mutation rate of between 0.80×10⁻³ and 2.38×10⁻³ nucleotide substitutions per site per year. In some embodiments, the virus has a mutation rate of between 1.16×10⁻³ and 3.30×10⁻³ non-synonymous nucleotide substitutions per site per year. In some embodiments, the virus has a mutation rate of between 1.67×10⁻³ and 4.67×10⁻³ synonymous nucleotide substitutions per site per year.

In some embodiments of the uses of the formulations of the disclosure, the virus presents antigenic drift.

In some embodiments of the uses of the formulations of the disclosure, a genomic sequence of the virus mutates or undergoes reassortment. In some embodiments, the genomic sequence of the virus mutates at a rate higher than 10⁻⁶ mutations per nucleotide of the genome per replication cycle. In some embodiments, the genomic sequence of the virus undergoes reassortment more frequently than 40% of total genomic reassortment.

In some embodiments of the uses of the formulations of the disclosure, the infection has an incubation period of between 1 and 15 days. In some embodiments, the infection has an incubation period of between 0.5 and 15 days. In some embodiments, the infection has an incubation period of between 0.5 and 1.5 days. In some embodiments, the infection has an incubation period of between 5 and 15 days.

In some embodiments of the uses of the formulations of the disclosure, the virus has a reproduction number (R0) of between 0.9 and 18. In some embodiments, the virus has a R0 of between 1.5 and 3.5. In some embodiments, the virus has a R0 of between 2 and 5.

In some embodiments of the uses of the formulations of the disclosure, the virus is an RNA virus. In some embodiments, the RNA virus is a positive-strand RNA virus. In some embodiments, the positive-strand RNA virus belongs to the family of Picornaviridae, Astroviridae, Caliciviridae, Hepeviridae Flaviviridae, Togaviridae, Arteriviridae, or Coronaviridae. In some embodiments, the positive-strand RNA virus belongs to the family of Coronaviridae. In some embodiments, the Coronaviridae virus is Bat coronavirus CDPHE15, Bat coronavirus HKU10, Rhinolophus ferrumequinum alphacoronavirus HuB-2013, Human coronavirus 229E, Lucheng Rn rat coronavirus, Ferret coronavirus, Mink coronavirus 1, Miniopterus bat coronavirus 1, Miniopterus bat coronavirus HKU8, Myotis ricketti alphacoronavirus Sax-2011, Nyctalus velutinus alphacoronavirus SC-2013, Porcine epidemic diarrhea virus, Scotophilus bat coronavirus 512, Rhinolophus bat coronavirus HKU2, Human coronavirus NL63, NL63-related bat coronavirus strain BtKYNL63-9b, Human coronavirus OC43, China Rattus coronavirus HKU24, Human coronavirus HKU1, Murine coronavirus-type species, Bat Hp-betacoronavirus Zhejiang2013, Hedgehog coronavirus 1, Middle East respiratory syndrome-related coronavirus (MERS-CoV), Pipistrellus bat coronavirus HKU5, Tylonycteris bat coronavirus HKU4, Rousettus bat coronavirus GCCDC1, Rousettus bat coronavirus HKU9, Severe acute respiratory syndrome-related coronavirus, Wigeon coronavirus HKU20, Bulbul coronavirus HKU11-type species, Porcine coronavirus HKU15, Munia coronavirus HKU13, White-eye coronavirus HKU16, Night heron coronavirus HKU19, Common moorhen coronavirus HKU21, Beluga whale coronavirus SW1 or Avian coronavirus-type species. In some embodiments, the Coronaviridae virus is Middle East respiratory syndrome-related coronavirus (MERS-CoV), Severe acute respiratory syndrome coronavirus (SARS-CoV) or Severe acute respiratory syndrome coronavirus 2 (SARS-CoV-2). In some embodiments, the Coronaviridae virus is Severe acute respiratory syndrome coronavirus 2 (SARS-CoV-2).

In some embodiments of the uses of the formulations of the disclosure, the RNA virus is a negative-strand RNA virus. In some embodiments, the negative-strand RNA virus belongs to the family of Orthomyxoviridae, Paramyxoviridae, Rhabdoviridiae, Filoviruses, or Arenaviruses. In some embodiments, the negative-strand virus is Influenza virus, Sendai virus, Human parainfluenza virus 1 (hPIV1), Simian virus 5 (SV5, PIV5), Mumps virus, Newcastle disease virus (NDV), Measles virus, Rinderpest virus, Respiratory syncytial virus (RSV), Vesicular stomatitis virus (VSV), Rabies virus, Ebola virus, Marburg virus, Lymphocytic choriomeningitis virus (LCMV), Junin virus, or Lassa fever virus.

In some embodiments of the uses of the formulations of the disclosure, the virus is a retrovirus. In some embodiments, the retrovirus is a Lentivirus, Alpharetrovirus, Betaretrovirus, Deltaretrovirus, Gammaretrovirus or Epsilonretrovirus. In some embodiments, the retrovirus is a Simian immunodeficiency virus (SIV), Human immunodeficiency virus-1 (HIV-1), HIV-2, Feline immunodeficiency virus (FIV), Equine infectious anemia virus (EIAV), Mouse mammary tumor-like virus (MMTV), Mason-Pfizer monkey virus (MPMV), Respiratory syncytial virus RSV, bovine leukemia virus (BLV), Human T-cell leukemia virus-1 (HTLV-1), HTLV-2, Murine leukemia virus (MuLV), Gibbon ape leukemia virus (GALV).

In some embodiments of the uses of the formulations of the disclosure, the virus is a DNA virus. In some embodiments, the DNA virus is Adenovirus, infectious canine hepatitis virus, Papillomavirus, polyomaviridae, simian vacuolating virus, Parvovirus B19, canine parvovirus, Herpes simplex virus, varicella-zoster virus, cytomegalovirus, Epstein-Barr virus, Smallpox virus, cow pox virus, sheep pox virus, monkey pox virus, vaccinia virus, Torque teno virus.

In some embodiments of the uses of the formulations of the disclosure, the subject has a medical condition that increases risk of a poor prognosis without treatment or an increased risk of a severe immune response to the infection. In some embodiments, the subject has an impaired or reduced immune response. In some embodiments, the subject is immunocompromised. In some embodiments, the subject is undergoing or has undergone one or more of an immunosuppressive therapy, presenting a sign or symptom of a second medical disease or disorder or a subject of a past, present or future medical intervention. In some embodiments, the second medical second medical disease or disorder comprises an infection, an HIV infection, an onset of Acquired Immunodeficiency Syndrome (AIDS), a proliferative disorder, a cancer, asthma or an autoimmune condition. In some embodiments, the immunosuppressive therapy comprises one or more of an ablation of bone marrow or circulating blood cell types, a radiation therapy, a therapy for a proliferative or malignant disease, a monoclonal antibody therapy, a blood transfusion, a bone marrow transplant, or removal of a lymph node. In some embodiments, the medical intervention comprises one or more of a surgery, a graft, a tissue or organ transplant, a wound or burn debridement, a setting of a bone, a replacement of a bone, a replacement of a joint, a cardiac bypass, a vascular stent placement, a spinal tap, an organ resection, or a cardiac pacemaker placement or replacement.

In some embodiments of the uses of the formulations of the disclosure, the subject presents a sign or symptom of hypertension or cardiac disease. In some embodiments, the subject has been diagnosed with hypertension or cardiac disease.

In some embodiments of the uses of the formulations of the disclosure, the subject presents a sign or symptom of a metabolic disorder. In some embodiments, the subject has been diagnosed with the metabolic disorder. In some embodiments, the metabolic disorder comprises insulin resistance, pre-diabetes, or diabetes.

In some embodiments of the uses of the formulations of the disclosure, the subject presents a sign or symptom of a clotting or vascular disorder. In some embodiments, the subject has been diagnosed with the clotting or vascular disorder. In some embodiments, the subject is at risk of forming a clot or suffering from a stroke.

In some embodiments of the uses of the formulations of the disclosure, the subject has not received a vaccine for the virus prior to contacting the virus or presenting the viral infection.

In some embodiments of the uses of the formulations of the disclosure, the subject is a neonatal subject. In some embodiments, the mother of the subject is a carrier of the virus or infected by the virus. In some embodiments, the subject is under 18 years of age. In some embodiments, the subject is between 18 years of age and 29 years of age, inclusive of the endpoints. In some embodiments, the subject is between 30 years of age and 49 years of age, inclusive of the endpoints. In some embodiments, the subject is between 50 years of age and 69 years of age, inclusive of the endpoints. In some embodiments, the subject is over 70 years of age.

The disclosure provides a method for treating or preventing a viral infection, comprising administering an effective amount of a formulation of the disclosure to a subject, wherein, upon contacting an infectious agent, the formulation prevents transmission, infection, replication, survival or growth of the virus in the subject.

In some embodiments of the methods of the disclosure, contacting an infectious agent comprises the subject contacting directly or indirectly an organism infected with the virus or contaminated by the virus. In some embodiments, the organism presents one or more sign(s) or symptom(s) of an infection. In some embodiments, the organism does not present a sign or a symptom of an infection. In some embodiments, the organism is an asymptomatic carrier of the virus.

In some embodiments of the methods of the disclosure, contacting comprises communication of the virus through air or through fluid media. In some embodiments, the fluid comprises a bodily fluid or particulate thereof from the infected or contaminated organism. In some embodiments, the bodily fluid particulate comprises an exhaled or an expelled droplet. In some embodiments, the bodily fluid particulate comprises an aerosolized droplet. In some embodiments, the bodily fluid comprises sputum, saliva, blood, plasma, serum, lymph fluid, tears, sweat, urine or feces.

In some embodiments of the methods of the disclosure, the virus is communicated to the subject from across a physical distance of between 0.1 and 12 feet from the organism. In some embodiments, the virus is communicated to the subject from across a physical distance of 6 feet or less from the organism.

In some embodiments of the methods of the disclosure, the virus survives, remains viable or retains an infectious activity on a surface for between 0.1 minute and 6 months. In some embodiments, the surface comprises a biological surface. In some embodiments, the surface comprises one or more of a plant, a tree, a crop, skin, hair, nails or a component thereof. In some embodiments, the surface comprises one or more of silk, cotton, cellulose, cork, wool, wood, cardboard, latex, rubber and paper.

In some embodiments of the methods of the disclosure, the virus survives, remains viable or retains an infectious activity on a surface for between 0.1 minute and 6 months. In some embodiments, the surface does not comprise a biological surface.

In some embodiments of the methods of the disclosure, the virus survives, remains viable or retains an infectious activity on a surface for between 0.1 minute and 6 months. In some embodiments, the surface comprises an organic surface. In some embodiments, the surface comprises one or more of carbon fiber, a plastic and synthetic fiber.

In some embodiments of the methods of the disclosure, the virus survives, remains viable or retains an infectious activity on a surface for between 0.1 minute and 6 months. In some embodiments, the surface comprises an inorganic surface. In some embodiments, the surface comprises one or more of a metal, silicone and glass.

In some embodiments of the methods of the disclosure, the organism infected with the virus or contaminated with the virus is a human.

In some embodiments of the methods of the disclosure, the organism infected with the virus or contaminated with the virus is not a human. In some embodiments, the organism is a mammalian organism, an avian organism, a reptilian organism, an amphibian organism, a crustacean organism, an arthropod organism or a chordata organism.

In some embodiments of the methods of the disclosure, the virus is a zoonotic virus.

In some embodiments of the methods of the disclosure, contacting comprises consumption or handling of the organism by the subject. In some embodiments, the organism is a domesticated animal. In some embodiments, the domesticated animal is a pet or an ornamental animal. In some embodiments, the pet or ornamental animal is a dog, a cat, a bird, a reptile or a rodent. In some embodiments, the rodent is a mouse, a rat, a rabbit, a hare or a hamster.

In some embodiments of the methods of the disclosure, contacting comprises consumption or handling of the organism by the subject. In some embodiments, the organism is a domesticated animal. In some embodiments, the organism is a live-stock animal.

In some embodiments of the methods of the disclosure, the organism is a wild animal.

In some embodiments of the methods of the disclosure, contacting occurs within a distance of 5 miles or less of: i) a human dwelling, ii) a laboratory or a research facility, iii) a market, store, or retail location, iv) a zoo, game reserve, wildlife reserve, land managed for wildlife protection or wildlife sanctuary, v) a farm, a field, or an agricultural location, vi) a hotel, a lodge, a resort or a site for an ecotourism activity, vii) a source of water, a well or a barrel maintained for drinking water, a stream, a river, a lake and an ocean, and/or viii) an airplane, a ship, a boat, a bus, a train, a car or a truck. In some embodiments, the organism is maintained in one or more of i) through viii).

In some embodiments of the methods of the disclosure, contacting the organism is intentional. In some embodiments, the organism is personal property, state property, communal property, a hunting target, a food source, a research subject, a pet, a native species, an invasive species, a prey of any one of the foregoing or a predator of any one of the foregoing.

In some embodiments of the methods of the disclosure, contacting the organism is unintentional. In some embodiments, the organism is a native species, an invasive species or a predator of the subject.

In some embodiments of the methods of the disclosure, the virus has a mutation rate of between 10⁻⁸ and 10⁻⁴ mutations per nucleotide of the genome per replication cycle.

In some embodiments of the methods of the disclosure, the virus has a mutation rate of between 10⁻⁵ and 10⁻² nucleotide substitutions per site per year. In some embodiments, the virus has a mutation rate of between 0.80×10⁻³ and 2.38×10⁻³ nucleotide substitutions per site per year. In some embodiments, the virus has a mutation rate of between 1.16×10⁻³ and 3.30×10⁻³ non-synonymous nucleotide substitutions per site per year. In some embodiments, the virus has a mutation rate of between 1.67×10⁻³ and 4.67×10⁻³ synonymous nucleotide substitutions per site per year.

In some embodiments of the methods of the disclosure, the virus presents antigenic drift.

In some embodiments of the methods of the disclosure, a genomic sequence of the virus mutates or undergoes reassortment. In some embodiments, the genomic sequence of the virus mutates at a rate higher than 10⁻⁶ mutations per nucleotide of the genome per replication cycle. In some embodiments, the genomic sequence of the virus undergoes reassortment more frequently than 40% of total genomic reassortment.

In some embodiments of the methods of the disclosure, the infection has an incubation period of between 1 and 15 days. In some embodiments, the infection has an incubation period of between 0.5 and 15 days. In some embodiments, the infection has an incubation period of between 0.5 and 1.5 days. In some embodiments, the infection has an incubation period of between 5 and 15 days.

In some embodiments of the methods of the disclosure, the virus has a reproduction number (R0) of between 0.9 and 18. In some embodiments, the virus has a R0 of between 1.5 and 3.5. In some embodiments, the virus has a R0 of between 2 and 5.

In some embodiments of the methods of the disclosure, the virus is an RNA virus. In some embodiments, the RNA virus is a positive-strand RNA virus. In some embodiments, the positive-strand RNA virus belongs to the family of Picornaviridae, Astroviridae, Caliciviridae, Hepeviridae Flaviviridae, Togaviridae, Arteriviridae, or Coronaviridae. In some embodiments, the positive-strand RNA virus belongs to the family of Coronaviridae. In some embodiments, the Coronaviridae virus is Bat coronavirus CDPHE15, Bat coronavirus HKU10, Rhinolophus ferrumequinum alphacoronavirus HuB-2013, Human coronavirus 229E, Lucheng Rn rat coronavirus, Ferret coronavirus, Mink coronavirus 1, Miniopterus bat coronavirus 1, Miniopterus bat coronavirus HKU8, Myotis ricketti alphacoronavirus Sax-2011, Nyctalus velutinus alphacoronavirus SC-2013, Porcine epidemic diarrhea virus, Scotophilus bat coronavirus 512, Rhinolophus bat coronavirus HKU2, Human coronavirus NL63, NL63-related bat coronavirus strain BtKYNL63-9b, Human coronavirus OC43, China Rattus coronavirus HKU24, Human coronavirus HKU1, Murine coronavirus-type species, Bat Hp-betacoronavirus Zhejiang2013, Hedgehog coronavirus 1, Middle East respiratory syndrome-related coronavirus (MERS-CoV), Pipistrellus bat coronavirus HKU5, Tylonycteris bat coronavirus HKU4, Rousettus bat coronavirus GCCDC1, Rousettus bat coronavirus HKU9, Severe acute respiratory syndrome-related coronavirus, Wigeon coronavirus HKU20, Bulbul coronavirus HKU11-type species, Porcine coronavirus HKU15, Munia coronavirus HKU13, White-eye coronavirus HKU16, Night heron coronavirus HKU19, Common moorhen coronavirus HKU21, Beluga whale coronavirus SW1 or Avian coronavirus-type species. In some embodiments, the Coronaviridae virus is Middle East respiratory syndrome-related coronavirus (MERS-CoV), Severe acute respiratory syndrome coronavirus (SARS-CoV) or Severe acute respiratory syndrome coronavirus 2 (SARS-CoV-2). In some embodiments, the Coronaviridae virus is Severe acute respiratory syndrome coronavirus 2 (SARS-CoV-2).

In some embodiments of the methods of the disclosure, the RNA virus is a negative-strand RNA virus. In some embodiments, the negative-strand RNA virus belongs to the family of Orthomyxoviridae, Paramyxoviridae, Rhabdoviridiae, Filoviruses, or Arenaviruses. In some embodiments, the negative-strand virus is Influenza virus, Sendai virus, Human parainfluenza virus 1 (hPIV1), Simian virus 5 (SV5, PIV5), Mumps virus, Newcastle disease virus (NDV), Measles virus, Rinderpest virus, Respiratory syncytial virus (RSV), Vesicular stomatitis virus (VSV), Rabies virus, Ebola virus, Marburg virus, Lymphocytic choriomeningitis virus (LCMV), Junin virus, or Lassa fever virus.

In some embodiments of the methods of the disclosure, the virus is a retrovirus. In some embodiments, the retrovirus is a Lentivirus, Alpharetrovirus, Betaretrovirus, Deltaretrovirus, Gammaretrovirus or Epsilonretrovirus. In some embodiments, the retrovirus is a Simian immunodeficiency virus (SIV), Human immunodeficiency virus-1 (HIV-1), HIV-2, Feline immunodeficiency virus (FIV), Equine infectious anemia virus (EIAV), Mouse mammary tumor-like virus (MMTV), Mason-Pfizer monkey virus (MPMV), Respiratory syncytial virus RSV, bovine leukemia virus (BLV), Human T-cell leukemia virus-1 (HTLV-1), HTLV-2, Murine leukemia virus (MuLV), Gibbon ape leukemia virus (GALV).

In some embodiments of the methods of the disclosure, the virus is a DNA virus. In some embodiments, the DNA virus is Adenovirus, infectious canine hepatitis virus, Papillomavirus, polyomaviridae, simian vacuolating virus, Parvovirus B19, canine parvovirus, Herpes simplex virus, varicella-zoster virus, cytomegalovirus, Epstein-Barr virus, Smallpox virus, cow pox virus, sheep pox virus, monkey pox virus, vaccinia virus, Torque teno virus.

In some embodiments of the methods of the disclosure, the subject has a medical condition that increases risk of a poor prognosis without treatment or an increased risk of a severe immune response to the infection. In some embodiments, the subject has an impaired or reduced immune response. In some embodiments, the subject is immunocompromised. In some embodiments, the subject is undergoing or has undergone one or more of an immunosuppressive therapy, presenting a sign or symptom of a second medical disease or disorder or a subject of a past, present or future medical intervention. In some embodiments, the second medical second medical disease or disorder comprises an infection, an HIV infection, an onset of Acquired Immunodeficiency Syndrome (AIDS), a proliferative disorder, a cancer, asthma or an autoimmune condition. In some embodiments, the immunosuppressive therapy comprises one or more of an ablation of bone marrow or circulating blood cell types, a radiation therapy, a therapy for a proliferative or malignant disease, a monoclonal antibody therapy, a blood transfusion, a bone marrow transplant, or removal of a lymph node. In some embodiments, the medical intervention comprises one or more of a surgery, a graft, a tissue or organ transplant, a wound or burn debridement, a setting of a bone, a replacement of a bone, a replacement of a joint, a cardiac bypass, a vascular stent placement, a spinal tap, an organ resection, or a cardiac pacemaker placement or replacement.

In some embodiments of the methods of the disclosure, the subject presents a sign or symptom of hypertension or cardiac disease. In some embodiments, the subject has been diagnosed with hypertension or cardiac disease.

In some embodiments of the methods of the disclosure, the subject presents a sign or symptom of a metabolic disorder. In some embodiments, the subject has been diagnosed with the metabolic disorder. In some embodiments, the metabolic disorder comprises insulin resistance, pre-diabetes, or diabetes.

In some embodiments of the methods of the disclosure, the subject presents a sign or symptom of a clotting or vascular disorder. In some embodiments, the subject has been diagnosed with the clotting or vascular disorder. In some embodiments, the subject is at risk of forming a clot or suffering from a stroke.

In some embodiments of the methods of the disclosure, the subject has not received a vaccine for the virus prior to contacting the virus or presenting the viral infection.

In some embodiments of the methods of the disclosure, the subject is a neonatal subject. In some embodiments, the mother of the subject is a carrier of the virus or infected by the virus. In some embodiments, the subject is under 18 years of age. In some embodiments, the subject is between 18 years of age and 29 years of age, inclusive of the endpoints. In some embodiments, the subject is between 30 years of age and 49 years of age, inclusive of the endpoints. In some embodiments, the subject is between 50 years of age and 69 years of age, inclusive of the endpoints. In some embodiments, the subject is over 70 years of age.

In some embodiments of the formulations for use in treating or preventing an infection, the subject is human. In some embodiments, the subject is female. In some embodiments, the subject is male. In some embodiments, the subject is a neonate. In some embodiments, the subject is an infant. In some embodiments, the subject is a child. In some embodiments, the subject is an adult. In some embodiments, the subject is a senior adult. In some embodiments, the subject is an elderly adult.

In some embodiments of the formulations for use in treating or preventing an infection, the subject is not admitted to a hospital or comparable facility to receive professional medical care. In some embodiments, the subject is admitted to a hospital or comparable facility to receive professional medical care.

In some embodiments of the formulations for use in treating or preventing an infection, the subject is not admitted to a residential care facility to receive routine medical care. In some embodiments, the subject is admitted to a residential care facility to receive routine medical care.

In some embodiments of the formulations for use in treating or preventing an infection, the subject is not connected to a ventilator. In some embodiments, the subject is connected to a ventilator. In some embodiments, the subject is in a coma. In some embodiments, the subject is not connected to a ventilator.

In some embodiments of the formulations for use in treating or preventing an infection, the subject is sentenced to a term or resident in a prison, jail, correctional facility or comparable facility.

In some embodiments of the formulations for use in treating or preventing an infection, the subject is deemed an essential worker by a governmental authority or an employer.

n some embodiments of the formulations for use in treating or preventing an infection, the subject has an increased risk of exposure to the virus or an increased risk of infection from the virus when compared to an average citizen. In some embodiments, the subject is one or more of an essential worker, an employee of a healthcare facility, an employee of an agricultural producer, an employee of a meat or diary processing plant, an employee of a food manufacturer, an employee of a food distributor, an employee of a food seller, an employee of a governmental agency, an employee of an emergency or essential agency, an emergency first-responder, an employee of a biological research, development or manufacturing company, an employee of a pharmacy, an employee of a warehouse owner, as student, a teacher and an employee of a funeral home.

The disclosure provides a method of increasing systemic bioavailability of niclosamide comprising mixing niclosamide with an amount of SLS that is at least about 3.0%, at least about 3.5%, at least about 4.0%, at least about 4.5%, at least about 5.0%, at least about 5.5%, at least about 6.0%, or at least about 6.5% weight by weight. In some embodiments, the method comprises mixing niclosamide with an amount of SLS that is at least 3.0%, at least 3.5%, at least 4.0%, at least 4.5%, at least 5.0%, at least 5.5%, at least 6.0%, or at least 6.5% weight by weight.

In some embodiments of the methods of making of the disclosure, the method further comprises mixing mannitol, MCC, Sodium Starch Glycolate, Hydroxypropyl cellulose (HPC), Colloidal Silicon Dioxide; and Sodium Stearyl Fumarate. In some embodiments, the method further comprises forming granules. In some embodiments, the method further comprises adding one or more extra-granular components. In some embodiments, the extra-granular components comprise: Sodium Starch Glycolate; Colloidal Silicon Dioxide; and Sodium Stearyl Fumarate. In some embodiments, the method further comprises forming a capsule. The disclosure provides a capsule prepared by a method of the disclosure.

### BRIEF DESCRIPTION OF THE DRAWINGS

FIG. 1 is a graph showing Luciferase kinetics of Vero E6 cells infected with nano luciferase reporter SARS-CoV-2. The cells were infected at a multiplicity of infection (MOI) of 0.3. Luciferase activities were measured at indicated time-points post-infection. The dotted line indicates background level of luciferase signal from cells without viral infection. Results from triplicate experiments were presented with bars representing standard deviations.

FIG. 2 is a graph showing Antiviral activity of niclosamide in cell culture. Vero E6 cells were infected with nano luciferase reporter SARS-CoV-2 (MOI of 0.1) in the presence of niclosamide. At 24 h post-infection, luciferase activities were measured to estimate the EC₅₀ value. The hill slope of the curve is also indicated. Results from triplicate experiments were presented with bars representing standard deviations.

FIG. 3 is a series of graphs depicting a first set of pharmacokinetic parameter projections for therapeutically-effective doses of niclosamide from the data of 5 representative patients (shown).

FIG. 4 is a graph and a table of a mean PK plot based on administration of 500 mg dose of niclosamide and a comparison (see table) against a first study using both 500 mg and 2000 mg doses and a second study using a 2000 mg dose.

FIG. 5 is a series of graphs depicting a first set of projected PK profiles compared to a first dosage study.

FIG. 6 is a series of graphs depicting a second set of projected PK profiles compared to a second dosage study.

FIG. 7 is a graph showing the dispersant properties of a talc formulation of niclosamide compared to a formulation containing an alternative excipient designed for use in solid formulations.

FIG. 8 is a chart providing exemplary formulations of the disclosure. The top row depicts the identity of the different formulations (blends) and the first column depicts the different components of the formulations, as indicated.

FIG. 9 is a chart providing exemplary formulations of the disclosure. The top row depicts the identity of the different formulations (blends) and the first column depicts the different components of the formulations, as indicated.

FIG. 10 is a graph showing the compressibility of the formulations from FIG. 9. The data illustrate a significant increase in compressibility with Formulation B. The x-axis depicts compression pressure and the y-axis depicts Tablet hardness.

FIG. 11 is a graph showing the bulk and tapped density of granules of the formulations from FIG. 9. The Y-axis depicts the trapped (empty bars) and bulk density (solid bars) (g/ml) and the x-axis indicates the different formulations tested, as indicated.

FIG. 12 is a graph showing the Hausner ratio physical mixtures and granules of the formulations from FIG. 9. All formulations demonstrated improved flow properties after compaction. The Y-axis depicts the Hausner ratio of the physical mixtures (empty bars) and granules (solid bars) and the y-axis indicates the different formulations tested, as indicated.

FIG. 13 is a graph showing the capsule fill weight of the different formulations, filled by granulated blend pouring v/s granulated blend tamping, as indicated. The y-axis depicts the weight in milligrams and the x-axis indicated the formulations.

FIG. 14 is a graph comparing the disintegration time of the pour and tamp filling capsules. The y-axis depicts the disintegration time and the x-axis depicts the formulations tested.

FIG. 15 is a chart showing the summary of the physical properties of the exemplary formulations evaluated in FIGS. 10-12.

FIGS. 16A-16J show the summary and characterization of exemplary formulations.

FIG. 16A is a chart summarizing the characteristics of a preferred formulation. The Top row depicts the individual formulations as indicated, and the first column depicts the characteristics as indicated. FIG. 16B is a chart showing the bulk density (g/ml) and trapped density (g/ml) of the formulations of FIG. 16A. FIG. 16C is a graph showing the net capsule filling weight of the formulations of FIG. 16A. The y-axis depicts the net capsule fill weight (mg) and the x-axis depicts the formulations. FIG. 16D is a series of graphs for showing the frequency of filling weight of the capsules for the different formulations of FIG. 16A, as indicated, along with the target filling weight. In each graph of the series, the y-axis depicts the frequency of filling weight and the x-axis indicates the filling weight achieved. FIG. 16E shows the disintegration time of the formulations of FIG. 16A. The y axis depicts the disintegration time in minutes and the x-axis depicts the formulations tested first time (empty bars) and second time (solid bars). FIG. 16F is a chart showing the time of disintegration of the formulations as indicated that were tested first and second time. FIG. 16G is a graph showing percentage drug release of the different formulations tested at Day 0 (top panel) and Day 7 (bottom panel) in 3.5% CTAB in phosphate buffer pH 6.8, as indicated. The y-axis depicts the percentage drug release and the x-axis depicts the time in minutes. FIG. 16 H is a series of charts showing the percentage release of the drug from the different formulations compared between Day 0 and Day 7. FIG. 16I is a graph showing the percentage release of the drug from the capsule batch #2200-007B tested on day 0, 1 week and 1 month. FIG. 16I is a chart showing the ranking of the different formulations tested in FIG. 16A in terms of the drug release rate at 90 minutes. Formulation A contains 5% SLS; while other formulations have 2.5% SLS. FIG. 16J shows that SLS content is key to release. SLS content is key to release.

FIG. 17 is a chart showing the final formulation. The top row depicts the concentration of the various components as percentage weight/weight (first column) and milligram per capsule (second column). The first column depicts the components of the final formulation. The amount of SLS was increased to 5% compared to the original Formulation B & adjusted with MCC. Formulation B is the robust for downstream processing and may be used for tablets.

FIG. 18 is a graph showing particle size distribution of the final blend depicted as cumulative percentage of powder (y-axis) v/s the particle size (μM) (x-axis).

FIG. 19 shows the concentration of niclosamide in human plasma after a single 1000 mg or 2000 mg oral dose of the final formulation.

### DETAILED DESCRIPTION

Antiviral

Niclosamide is formulated according to the disclosure for use as a monotherapy or as a combination therapy with any other second agent for the treatment of a viral infection. Without wishing to be bound by theory, niclosamide can disrupt, decrease or inhibit the transcription or translation of viral sequences in a host cell, thereby preventing a virus from reproducing itself inside of an infected host. One obstacle for the development of niclosamide as an anti-viral formulation suitable for systemic administration to treat any viral infection is the pharmacokinetic profile of the niclosamide in vivo following administration. The disclosure provides not only improved formulations, but also for existing formulations of niclosamide suitable for systemic administration, a new dosing schedule to provide or maintain a sufficient amount of niclosamide in the subject's bloodstream, increasing both bioavailability and biodistribution, to effectively treat a viral infection.

As previously approved by the US FDA, a dry solid formulation of niclosamide available as a compressed pill, was administered at a dose of 2 grams or 2000 mg once per day for the treatment of a parasitic infection that was local to the gastrointestinal tract.

The disclosure provides an alternative dosage schedule of either 1 gram or 1000 mg provided twice or three times per day, or as a higher dose, 2 grams or 2000 mg provided twice per day. For example, the more frequent dosing, even at a lower dosage per administration, maintains, on average a blood concentration and timing profile for the niclosamide that is safe and effective for use as an antiviral.

### Coronavirus

Coronavirus are a particularly interesting target of the formulations and methods of the disclosure. For clarity, the formulations and methods of the disclosure are not intended for the exclusive use of a coronavirus infection. However, several hallmarks of the coronavirus and the outbreaks of related viruses render the formulations and methods of the disclosure particularly useful or effective.

Coronaviruses are particularly successful at becoming a seasonal infection or spreading under a pandemic model of disease. This is due, in part, to the fact that a successfully spread virus does not immediately kill its host. Large portions of a population may be infected before showing a sign or symptom of infection and therefore, may be carriers of the virus for a relatively long time (as opposed to a more deadly infection). Coronavirus are often respiratory in nature and become air-borne which contributes to their infectivity rate. Compared to other viruses known to cause pandemics, like influenza, the coronavirus has a slower rate of mutation and produces fewer alternative strains. While this appears to be an advantage for the human population at first, this slower mutation rate and greater nucleotide proofreading ability decreases the likelihood of unsuccessful strains proliferating and competing with highly virulent strains. In other words, unlike the influenza pandemic of 1918, a coronavirus pandemic is less likely to compete itself out or kill so many hosts that it runs out of new carriers.

Because niclosamide is not specific for anyone virus, and because it has been shown to be safe for systemic administration by previous studies submitted to regulatory authorities, niclosamide formulations of the disclosure are an optimal first-line therapy for any viral infection, including any virus with a potential for causing a pandemic because it can be administered before the novel virus is fully sequenced or characterized for the development of a vaccine.

### SARS-Cov-2 Synergy

Niclosamide has been used clinically as a treatment for tapeworm infection. A dry solid formulation of Niclosamide has been previously approved by the United States Federal Drug Administration (FDA) for this indication. The disclosure provides improved dry solid formulations of niclosamide that control drug release, provide superior pharmacokinetics, and improve one or more of the stability, biodistribution and bioavailability of the niclosamide for the treatment of a viral infection.

Niclosamide modulates various cellular signaling pathways. In addition to a function uncoupling of mitochondrial oxidative phosphorylation, Niclosamide modulates Wnt/β-catenin, mTORC1, STATS, NF-κB and Notch signaling pathways. Due to its involvement with multiple signaling pathways, niclosamide can improve a sign or symptom of an underlying condition that renders a viral infection, including a coronavirus infection, more dangerous. Because niclosamide has potent antiviral activities, including potent antiviral activities against coronavirus strains, the use of niclosamide provide a synergistic benefit to a subject treated for a viral infection having one or more underlying conditions of the disclosure.

Niclosamide or its salt form, niclosamide ethanolamine, may be used as a monotherapy or a combination therapy in the treatment for one or more of: a bacterial infection (e.g. often occur concomitantly with viral infections); a parasitic infection (which can weaken the immune system); asthma (one risk factor to be infected with SARS-CoV-2); type II diabetes (one risk factor to be infected with SARS-CoV-2); cancer (e.g. prostate, cervical, colon) (chemotherapy and radiation therapy suppress or weaken the immune system); sclerodermatous graft-versus-host disease (requires immunosuppressive therapy); neuropathic pain (the use of non-steroidal anti-inflammatory drugs (NSAIDs) may weaken the immune system); rheumatoid arthritis (NSAIDs and steroids have been used as treatment, which can weaken the immune system); nonalcoholic steatohepatitis (NASH) (underlying condition is obesity and very likely also type II diabetes); nonalcoholic fatty liver disease (NAFLD) (underlying condition is obesity and very likely also type II diabetes); artery constriction (blood clotting is one result of COVID-19, which might be less severe in dilated blood vessels).

A subject of the disclosure having a viral infection and one or more of a bacterial infection; a parasitic infection; diabetes; Type I diabetes; type II diabetes; cancer; sclerodermatous graft-versus-host disease; neuropathic pain; rheumatoid arthritis; nonalcoholic steatohepatitis (NASH); nonalcoholic fatty liver disease (NAFLD) and artery constriction or a subject receiving a medical intervention of the treatment of one or more of a bacterial infection; a parasitic infection; diabetes; Type I diabetes; type II diabetes; cancer; sclerodermatous graft-versus-host disease; neuropathic pain; rheumatoid arthritis; nonalcoholic steatohepatitis (NASH); nonalcoholic fatty liver disease (NAFLD) and artery constriction may experience a synergistic benefit from simultaneous treatment of the viral infection and the underling condition following administration of a niclosamide formulation of the disclosure.

### Niclosamide

Formulations of the disclosure comprise an amount of niclosamide or pharmaceutically acceptable salt thereof.

Niclosamide or pharmaceutically acceptable salt thereof has the chemical formula: C₁₃H₈Cl₂N₂O₄, molecular weight of 327.12 g/mol and the chemical structure:

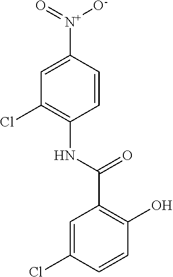 [SMILES: O=C(Nc1ccc([N+](=O)[O-])cc1Cl)c1cc(Cl)ccc1O]

The use of Nicosamide had been approved by the United States Federal Drug Administration (FDA) under the trade name “NICLOCIDE” as an orally administrated chewable tablet having 500 mg of the niclosamide active ingredient for the treatment of tapeworm infections. Niclocide has been discontinued, although alternative formulations of niclosamide are on the market for this indication.

### Niclosamide Formulations

Formulations of the disclosure, including a solid or a dry solid form may comprise Niclosamide or pharmaceutically acceptable salt thereof.

Formulations of the disclosure may comprise an amount of niclosamide, wherein the niclosamide comprises a dry and solid form, and one or more of an excipient, a pH modulator, a preservative, a binder, and a filler.

Exemplary excipients of the disclosure include, but are not limited to, Microcrystalline Cellulose, Hydroxypropyl Methylcellulose, Crospovidone (CPVD; disintegrant) or any combination thereof. In some embodiments, MCC is used as one or more of an excipient, a filler, a binder and a disintegrant. In some embodiments, HPMC is used as an excipient or a binder. In some embodiments, CPVD is used as an excipient or a distintegrant.

In some embodiments, the formulations of the disclosure, comprise a preservative. In some embodiments, the preservative comprise one or more of benzyl alcohol, chlorhexidine gluconate and benzoic acid. In some embodiments, the preservative comprise chlorhexidine gluconate.

In some embodiments, the formulations of the disclosure, comprise a preservative that is any one or more of vitamin A, Vitamin C, Vitamin E, retinyl palmitate, methionine, BHA (butylatedhydroxyanisole), BHT (Butylatedhydroxytoulene), selenium, cysteine propyl gallate, phenol, parabens including but not limited to Ethyl Paraben, methyl paraben, Propyl Paraben, Butyl Paraben, EDTA, Citric acid, sodium citrate, Benzyl Alcohol, Chlorobutanol, Meta cresol, Chloro cresol, Benzoic acid, Sorbic acid, Thiomersal, Bronopol Diols, Propylene Glycol, Benzylkonium Chloride, Benzethonium Chloride, Chlorhexidine Gluconate and Benzoic acid. In some embodiments, the formulations of the disclosure, comprise a preservative that is a combination of Benzyl Alcohol, Chlorhexidine Gluconate and Benzoic Acid.

The disclosure provides a formulation comprising an amount of niclosamide, wherein the niclosamide comprises a dry and solid form, and one or more of one or more of a surfactant, an excipient, a pH modulator, a preservative, a binder, a glidant, a dispersant, a disintegrant and a filler. In some embodiments, the glidant or the dispersant comprises talc. In some embodiments, the formulation comprises niclosamide and one or more of a glidant and a dispersant. In some embodiments, the formulation comprises niclosamide and one or more of a glidant and a dispersant, wherein the glidant or the dispersant comprises talc. In some embodiments, the formulation comprises niclosamide and one or more of a glidant and a dispersant in a ratio of 2:1. In some embodiments, the formulation comprises niclosamide and one or more of a glidant and a dispersant in a ratio of 2:1, wherein the glidant or the dispersant comprises talc.

In some embodiments of the formulations of the disclosure, the formulation comprises a plurality of nanospheres, a plurality of microspheres, a plurality of particulates, a powder, a pill, a capsule, or a tablet. In some embodiments, the niclosamide comprises a plurality of nanospheres, a plurality of microspheres, a plurality of particulates, a powder, a pill, a capsule, or a tablet. In some embodiments, the one or more of the surfactant, the excipient, the pH modulator, the preservative, the binder, the glidant, the dispersant, the disintegrant and the filler comprises a plurality of nanospheres, a plurality of microspheres, a plurality of particulates, a powder, a pill, a capsule, or a tablet.

In some embodiments of the formulations of the disclosure, the formulation comprises one or more compartment(s). In some embodiments, the one or more compartment(s) comprise at least two compartments and wherein the at least two compartments are configured to comprise at least one inner compartment and at least one outer compartment. In some embodiments, the at least one inner compartment or the at least one outer compartment comprises a controlled-release formulation. In some embodiments, the at least one inner compartment or the at least one outer compartment comprises a sustained-release formulation. In some embodiments, the at least one inner compartment or the at least one outer compartment comprises an immediate-release formulation.

In some embodiments of the formulations of the disclosure, the excipient or the surfactant increases the permeability of a tissue within the subject to the niclosamide. In some embodiments, the tissue comprises an endothelial membrane. In some embodiments, a component of the intestinal tract or the stomach comprises the tissue. In some embodiments, a component of the respiratory tract comprises the tissue. In some embodiments, a component of the blood brain barrier comprises the tissue.

In some embodiments of the formulations of the disclosure, one or more of the surfactant, the excipient, the pH modulator, the preservative, the binder, the glidant, the dispersant, the disintegrant and the filler increase the in vivo stability of the formulation or of the niclosamide.

In some embodiments of the formulations of the disclosure, one or more of the surfactant, the excipient, the pH modulator, the preservative, the binder, the glidant, the dispersant, the disintegrant and the filler increase the shelf life or in vitro stability of the formulation or of the niclosamide.

In some embodiments of the formulations of the disclosure, one or more of the surfactant, the excipient, the pH modulator, the preservative, the binder, the glidant, the dispersant, the disintegrant and the filler increase the biodistribution of the formulation or of the niclosamide.

In some embodiments of the formulations of the disclosure, one or more of the surfactant, the excipient, the pH modulator, the preservative, the binder, the glidant, the dispersant, the disintegrant and the filler maintain a constant circulating blood plasma concentration of the formulation or of the niclosamide.

In some embodiments of the formulations of the disclosure, a liquid or a suspension comprises the formulation or the niclosamide. In some embodiments, the formulation is provided in a unit dosage form. In some embodiments, the unit dosage form comprises a single dose of the formulation and wherein a single dose comprises an amount of niclosamide effective to reduce, partially or completely, a risk of infection by a virus. In some embodiments, the unit dosage form comprises at least two doses of the formulation and wherein a single dose comprises an amount of niclosamide effective to reduce, partially or completely, a risk of infection by a virus.

In some embodiments of the formulations of the disclosure, a food or a beverage comprises the formulation. In some embodiments, the combination of the food or the beverage and the formulation forms a suspension. In some embodiments, the combination of the food or the beverage and the formulation forms a liquid. In some embodiments, the formulation dissolves in the food or the beverage. In some embodiments, the food or the beverage comprises one or more of a dietary supplement, a meal replacement, an electrolyte, a protein, a sugar and a pharmaceutical carrier. In some embodiments, the food or the beverage comprises a pharmaceutical carrier and wherein one or more of an intravenous tube, a feeding tube or a central line comprise the formulation. In some embodiments, the pharmaceutical carrier or the formulation comprises one or more of a nanoparticle or a polymer. In some embodiments, the food or the beverage comprises a pediatric formula, a diabetic formula or a senior formula. In some embodiments, the food or the beverage comprises caffeine. In some embodiments, the food or the beverage comprises alcohol.

In some embodiments of the formulations of the disclosure, the formulation is suitable for systemic administration. In some embodiments, the systemic administration comprises administration via an oral, inhaled, or intravenous route.

In some embodiments of the formulations of the disclosure, including those in which the formulation is suitable for systemic administration, the formulation comprises a dry powder and wherein the formulation is nebulized, aerosolized, or vaporized for inhaled systemic administration. In some embodiments, the niclosamide formulation is suitable for use or provided for use in a nebulizer as a nebulized formulation. In some embodiments, the niclosamide formulation is suitable for use or provided for use in a humidifier or inhaler as a droplet or aerosolized formulation. In some embodiments, the niclosamide formulation is suitable for use or provided for use in a humidifier or inhaler as a droplet or an aerosolized formulation. In some embodiments, the niclosamide formulation is suitable for use or provided for use in a vaporizer a vaporized formulation. In some embodiments, the vaporized formulation is suitable for use or provided for use in a vaporizing device, including, but not limited to a vaporizer or vapor pen (also referred to as a vape pen).

In some embodiments of the formulations of the disclosure, including those in which the formulation is suitable for systemic administration, the formulation comprises a dry powder and wherein the formulation is solubilized for injected systemic administration.

In some embodiments of the formulations of the disclosure, the niclosamide comprises at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 80%, at least 95%, at least 97%, at least 99% or at least any percentage in between weight of niclosamide by weight of the formulation. In some embodiments, the niclosamide comprises about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 80%, about 95%, about 97%, about 99% or about any percentage in between weight of niclosamide by weight of the formulation. In some embodiments, the niclosamide comprises 1%, 5%, 10%, 15%, 20%, 25%, 30%, 35%, 40%, 45%, 50%, 55%, 60%, 65%, 70%, 75%, 80%, 85%, 80%, 95%, 97%, 99% or any percentage in between weight of niclosamide by weight of the formulation.

In some embodiments of the formulations of the disclosure, the formulation comprises niclosamide comprising between 0.1 and 5 grams of niclosamide. In some embodiments, the formulation comprises niclosamide comprising 500 milligrams (mg) of niclosamide. In some embodiments, the formulation comprises niclosamide comprising 1 gram of niclosamide. In some embodiments, the formulation comprises niclosamide comprising 2 grams of niclosamide.

In some embodiments of the formulations of the disclosure, the formulation comprises one or more of mannitol, microcrystalline cellulose (MCC), sodium lauryl sulfate (SLS), Sodium Starch Glycolate, Hydroxypropyl cellulose (HPC), Colloidal Silicon Dioxide, and Sodium Stearyl Fumarate. In some embodiments, the niclosamide comprises between 50% and 60% weight by weight of the formulation. In some embodiments, the niclosamide comprises between 54% and 57% weight by weight of the formulation. In some embodiments, the niclosamide comprises between 55% and 56% weight by weight of the formulation. In some embodiments, the niclosamide comprises 55% weight by weight of the formulation. In some embodiments, the niclosamide comprises 55.6% weight by weight of the formulation. In some embodiments, the formulation comprises mannitol and wherein the mannitol comprises between 4% and 12% weight by weight of the formulation. In some embodiments, the formulation comprises mannitol and wherein the mannitol comprises between 6% and 10% weight by weight of the formulation. In some embodiments, the formulation comprises mannitol and wherein the mannitol comprises 8% weight by weight of the formulation. In some embodiments, the formulation comprises mannitol and wherein the mannitol comprises 8.8% weight by weight of the formulation. In some embodiments, the formulation comprises MCC and wherein the MCC comprises between 12% and 18% weight by weight of the formulation. In some embodiments, the formulation comprises MCC and wherein the MCC comprises between 14% and 16% weight by weight of the formulation. In some embodiments, the formulation comprises MCC and wherein the MCC comprises 15% weight by weight of the formulation. In some embodiments, the formulation comprises MCC and wherein the MCC comprises 15.1% weight by weight of the formulation. In some embodiments, the formulation comprises SLS and wherein the SLS comprises at least 3% weight by weight of the formulation. In some embodiments, the formulation comprises SLS and wherein the SLS comprises between 3% and 7% weight by weight of the formulation. In some embodiments, the formulation comprises SLS and wherein the SLS comprises between 4% and 6% weight by weight of the formulation. In some embodiments, the formulation comprises SLS and wherein the SLS comprises 5% weight by weight of the formulation. In some embodiments, the formulation comprises Sodium Starch Glycolate and wherein the Sodium Starch Glycolate comprises between 4% and 12% weight by weight of the formulation. In some embodiments, the formulation comprises Sodium Starch Glycolate and wherein the Sodium Starch Glycolate comprises between 7% and 9% weight by weight of the formulation. In some embodiments, the formulation comprises Sodium Starch Glycolate and wherein the Sodium Starch Glycolate comprises 8% weight by weight of the formulation. In some embodiments, the formulation comprises HPC and wherein the HPC comprises between 3% and 7% weight by weight of the formulation. In some embodiments, the formulation comprises HPC and wherein the HPC comprises between 4% and 6% weight by weight of the formulation. In some embodiments, the formulation comprises HPC and wherein the HPC comprises 5% weight by weight of the formulation. In some embodiments, the formulation comprises Colloidal Silicon Dioxide and wherein the Colloidal Silicon Dioxide comprises between 0.1% and 0.9% weight by weight of the formulation. In some embodiments, the formulation comprises Colloidal Silicon Dioxide and wherein the Colloidal Silicon Dioxide comprises between 0.4% and 0.6% weight by weight of the formulation. In some embodiments, the formulation comprises Colloidal Silicon Dioxide and wherein the Colloidal Silicon Dioxide comprises 0.5% weight by weight of the formulation. In some embodiments, the formulation comprises Sodium Stearyl Fumarate and wherein the Sodium Stearyl Fumarate comprises between 0.1% and 4% weight by weight of the formulation. In some embodiments, the formulation comprises Sodium Stearyl Fumarate and wherein the Sodium Stearyl Fumarate comprises between 1% and 3% weight by weight of the formulation. In some embodiments, the formulation comprises Sodium Stearyl Fumarate and wherein the Sodium Stearyl Fumarate comprises 2% weight by weight of the formulation.

In some embodiments of the formulations of the disclosure, the formulation comprises one or more of mannitol, microcrystalline cellulose (MCC), sodium lauryl sulfate (SLS), Sodium Starch Glycolate, Hydroxypropyl cellulose (HPC), Colloidal Silicon Dioxide, and Sodium Stearyl Fumarate. In some embodiments, the niclosamide comprises between about 50% and about 60% weight by weight of the formulation. In some embodiments, the niclosamide comprises between about 54% and about 57% weight by weight of the formulation. In some embodiments, the niclosamide comprises between about 55% and about 56% weight by weight of the formulation. In some embodiments, the niclosamide comprises about 55% weight by weight of the formulation. In some embodiments, the niclosamide comprises about 55.6% weight by weight of the formulation. In some embodiments, the formulation comprises mannitol and wherein the mannitol comprises between about 4% and about 12% weight by weight of the formulation. In some embodiments, the formulation comprises mannitol and wherein the mannitol comprises between about 6% and about 10% weight by weight of the formulation. In some embodiments, the formulation comprises mannitol and wherein the mannitol comprises about 8% weight by weight of the formulation. In some embodiments, the formulation comprises mannitol and wherein the mannitol comprises about 8.8% weight by weight of the formulation. In some embodiments, the formulation comprises MCC and wherein the MCC comprises between about 12% and about 18% weight by weight of the formulation. In some embodiments, the formulation comprises MCC and wherein the MCC comprises between about 14% and about 16% weight by weight of the formulation. In some embodiments, the formulation comprises MCC and wherein the MCC comprises about 15% weight by weight of the formulation. In some embodiments, the formulation comprises MCC and wherein the MCC comprises about 15.1% weight by weight of the formulation. In some embodiments, the formulation comprises SLS and wherein the SLS comprises at least about 3% weight by weight of the formulation. In some embodiments, the formulation comprises SLS and wherein the SLS comprises between about 3% and about 7% weight by weight of the formulation. In some embodiments, the formulation comprises SLS and wherein the SLS comprises between about 4% and about 6% weight by weight of the formulation. In some embodiments, the formulation comprises SLS and wherein the SLS comprises about 5% weight by weight of the formulation. In some embodiments, the formulation comprises Sodium Starch Glycolate and wherein the Sodium Starch Glycolate comprises between about 4% and about 12% weight by weight of the formulation. In some embodiments, the formulation comprises Sodium Starch Glycolate and wherein the Sodium Starch Glycolate comprises between about 7% and about 9% weight by weight of the formulation. In some embodiments, the formulation comprises Sodium Starch Glycolate and wherein the Sodium Starch Glycolate comprises about 8% weight by weight of the formulation. In some embodiments, the formulation comprises HPC and wherein the HPC comprises between about 3% and about 7% weight by weight of the formulation. In some embodiments, the formulation comprises HPC and wherein the HPC comprises between about 4% and about 6% weight by weight of the formulation. In some embodiments, the formulation comprises HPC and wherein the HPC comprises about 5% weight by weight of the formulation. In some embodiments, the formulation comprises Colloidal Silicon Dioxide and wherein the Colloidal Silicon Dioxide comprises between about 0.1% and about 0.9% weight by weight of the formulation. In some embodiments, the formulation comprises Colloidal Silicon Dioxide and wherein the Colloidal Silicon Dioxide comprises between about 0.4% and about 0.6% weight by weight of the formulation. In some embodiments, the formulation comprises Colloidal Silicon Dioxide and wherein the Colloidal Silicon Dioxide comprises about 0.5% weight by weight of the formulation. In some embodiments, the formulation comprises Sodium Stearyl Fumarate and wherein the Sodium Stearyl Fumarate comprises between about 0.1% and about 4% weight by weight of the formulation. In some embodiments, the formulation comprises Sodium Stearyl Fumarate and wherein the Sodium Stearyl Fumarate comprises between about 1% and about 3% weight by weight of the formulation. In some embodiments, the formulation comprises Sodium Stearyl Fumarate and wherein the Sodium Stearyl Fumarate comprises about 2% weight by weight of the formulation.

The disclosure provides a formulation comprising (a) niclosamide, comprising 55% weight by weight of the formulation; (b) mannitol, comprising 8% weight by weight of the formulation; (c) MCC, comprising 15% weight by weight of the formulation; (d) SLS, comprising 5% weight by weight of the formulation; (e) Sodium Starch Glycolate, comprising 8% weight by weight of the formulation; (f) Hydroxypropyl cellulose (HPC), comprising 5% weight by weight of the formulation; (g) Colloidal Silicon Dioxide, comprising 0.5% weight by weight of the formulation; and (h) Sodium Stearyl Fumarate, comprising 2% weight by weight of the formulation.

The disclosure provides a formulation comprising (a) niclosamide, comprising about 55% to about 56% weight by weight of the formulation; (b) mannitol, comprising about 8% to about 9% weight by weight of the formulation; (c) MCC, comprising about 15% to about 16% weight by weight of the formulation; (d) SLS, comprising about 5% weight by weight of the formulation; (e) Sodium Starch Glycolate, comprising about 8% weight by weight of the formulation; (f) Hydroxypropyl cellulose (HPC), comprising about 5% weight by weight of the formulation; (g) Colloidal Silicon Dioxide, comprising about 0.5% weight by weight of the formulation; and (h) Sodium Stearyl Fumarate, comprising about 2% weight by weight of the formulation.

The disclosure provides a formulation comprising (a) 250 mg of niclosamide; (b) 39.6 mg of mannitol, (c) 68.1 mg of MCC; (d) 22.5 mg of SLS; (e) 36 mg of Sodium Starch Glycolate; (f) 22.5 mg of Hydroxypropyl cellulose (HPC); (g) 2.3 mg of Colloidal Silicon Dioxide; and (h) 9 mg of Sodium Stearyl Fumarate.

The disclosure provides a formulation comprising (a) about 250 mg of niclosamide; (b) about 39.6 mg of mannitol, (c) about 68.1 mg of MCC; (d) about 22.5 mg of SLS; (e) about 36 mg of Sodium Starch Glycolate; (f) about 22.5 mg of Hydroxypropyl cellulose (HPC); (g) about 2.3 mg of Colloidal Silicon Dioxide; and (h) about 9 mg of Sodium Stearyl Fumarate.

The disclosure provides a formulation comprising (i) intra-granular components and (ii) extra-granular components; wherein the intra-granular components comprise: (a) niclosamide, comprising about 55% to about 56% weight by weight of the formulation; (b) mannitol, comprising about 8% to about 9% weight by weight of the formulation; (c) MCC, comprising about 15% to about 16% weight by weight of the formulation; (d) SLS, comprising about 5% weight by weight of the formulation; (e) Sodium Starch Glycolate, comprising about 5% weight by weight of the formulation; (f) Hydroxypropyl cellulose (HPC), comprising about 5% weight by weight of the formulation; (g) Colloidal Silicon Dioxide, comprising about 0.3% weight by weight of the formulation; and (h) Sodium Stearyl Fumarate, comprising about 1% weight by weight of the formulation; and wherein the extra-granular components comprise: (i) Sodium Starch Glycolate, comprising about 3% weight by weight of the formulation; (j) Colloidal Silicon Dioxide, comprising about 0.2% weight by weight of the formulation; and (k) Sodium Stearyl Fumarate, comprising about 1% weight by weight of the formulation.

The disclosure provides a formulation comprising (i) intra-granular components and (ii) extra-granular components; wherein the intra-granular components comprise: (a) niclosamide, comprising about 55.6% weight by weight of the formulation; (b) mannitol, comprising about 8.8% weight by weight of the formulation; (c) MCC, comprising about 15.1% weight by weight of the formulation; (d) SLS, comprising about 5% weight by weight of the formulation; (e) Sodium Starch Glycolate, comprising about 5% weight by weight of the formulation; (f) Hydroxypropyl cellulose (HPC), comprising about 5% weight by weight of the formulation; (g) Colloidal Silicon Dioxide, comprising about 0.3% weight by weight of the formulation; and (h) Sodium Stearyl Fumarate, comprising about 1% weight by weight of the formulation; and wherein the extra-granular components comprise: (i) Sodium Starch Glycolate, comprising about 3% weight by weight of the formulation; (j) Colloidal Silicon Dioxide, comprising about 0.2% weight by weight of the formulation; and (k) Sodium Stearyl Fumarate, comprising about 1% weight by weight of the formulation.

The disclosure provides a formulation comprising (i) intra-granular components and (ii) extra-granular components; wherein the intra-granular components comprise: (a) about 250 mg of niclosamide; (b) about 39.6 mg of mannitol; (c) about 68.1 mg of MCC; (d) about 22.5 mg of SLS; (e) about 22.5 mg of Sodium Starch Glycolate; (f) about 22.5 mg of Hydroxypropyl cellulose (HPC); (g) about 1.4 mg of Colloidal Silicon Dioxide; and (h) about 4.5 mg of Sodium Stearyl Fumarate; and wherein the extra-granular components comprise: (i) about 13.5 mg of Sodium Starch Glycolate; (j) about 0.9 mg of Colloidal Silicon Dioxide; and (k) about 4.5. mg of Sodium Stearyl Fumarate.

In some embodiments of the formulations of the disclosure, the formulation is in a capsule.

In some embodiments of the formulations of the disclosure, the formulation is not a tablet.

In some embodiments of the formulations of the disclosure, the formulation shows a higher percentage of drug release than a reference formulation comprising 500 mg niclosamide, maize starch, talcum, sodium lauryl sulphate, povidone, vanillin, magnesium stearate, and saccharin sodium. In some embodiments, the formulation provides a higher percentage of drug release than a percentage of drug release of a reference formulation. In some embodiments, the reference formulation comprises 500 mg niclosamide, maize starch, talcum, sodium lauryl sulphate, povidone, vanillin, magnesium stearate, and saccharin sodium. In some embodiments, the reference formulation comprises a tablet comprising 500 mg niclosamide, maize starch, talcum, sodium lauryl sulphate, povidone, vanillin, magnesium stearate, and saccharin sodium.

In some embodiments of the formulations of the disclosure, the formulation shows higher percentage of drug release than a reference formulation comprising niclosamide and an amount of SLS that is less than 2.5% weight by weight of the formulation. In some embodiments, the formulation provides a higher percentage of drug release than a percentage of drug release from a reference formulation. In some embodiments, the reference formulation comprises 500 mg niclosamide, maize starch, talcum, sodium lauryl sulphate, povidone, vanillin, magnesium stearate, and saccharin sodium. In some embodiments, the reference formulation comprises a tablet comprising 500 mg niclosamide, maize starch, talcum, sodium lauryl sulphate, povidone, vanillin, magnesium stearate, and saccharin sodium.

In some embodiments of the formulations of the disclosure, the formulation has systemic bioavailability higher than about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% when administered orally. In some embodiments, the formulation provides a systemic bioavailability of equal to or greater than 10%, 15%, 20%, 25%, 30%, 35%, 40%, 45%, 50%, 55%, 60%, 65%, 70%, 75%, 80%, 85%, 90%, 95%, 99% or any percentage in between. In some embodiments, the formulation has higher systemic bioavailability than a reference formulation comprising 500 mg niclosamide, maize starch, talcum, sodium lauryl sulphate, povidone, vanillin, magnesium stearate, and saccharin sodium. In some embodiments, the formulation has a higher systemic bioavailability than the systemic bioavailability of a reference formulation. In some embodiments, the reference formulation comprises 500 mg niclosamide, maize starch, talcum, sodium lauryl sulphate, povidone, vanillin, magnesium stearate, and saccharin sodium. In some embodiments, the reference formulation comprises a tablet comprising 500 mg niclosamide, maize starch, talcum, sodium lauryl sulphate, povidone, vanillin, magnesium stearate, and saccharin sodium.

In some embodiments of the formulations of the disclosure, including those formulations comprising one or more of mannitol, microcrystalline cellulose (MCC), sodium lauryl sulfate (SLS), Sodium Starch Glycolate, Hydroxypropyl cellulose (HPC), Colloidal Silicon Dioxide, and Sodium Stearyl Fumarate, the formulation comprises a plurality of spheres. In some embodiments, the formulation comprises a plurality of microspheres. In some embodiments, the formulation comprises a dry or solid particulate, optionally, compressed into a plurality of spheres or microspheres. In some embodiments, the formulation comprises a dry or solid particulate, optionally, compressed into a plurality of spheres or microspheres, wherein the plurality of spheres or microspheres are enclosed or encapsulated. In some embodiments, the formulation comprises a capsule comprising a dry or solid particulate, optionally, compressed into a plurality of spheres or microspheres. In some embodiments, the capsule is administered orally. In some embodiments, the capsule is broken, opened or separated to release the dry or solid particulate, optionally, compressed into a plurality of spheres or microspheres into a liquid to generate a liquid or a suspended formulation. In some embodiments, the liquid or the suspended formulation is administered orally (oral route), intravenously (intravenous route), intrajejunally (intrajejunal route), intrathecally (intrathecal route), intraspinally (intraspinal route). In some embodiments, the liquid or the suspended formulation is dispersed as wet formulation. In some embodiments, the liquid or the suspended formulation is atomized, vaporized, nebulized, aerosolized, or otherwise dispersed as a wet formulation for inhaled or intranasal administration.

In some embodiments, the formulations of the disclosure are pharmaceutical formulations. Pharmaceutical formulations comprise one or more pharmaceutically-acceptable carriers.

In some embodiments, the formulations of the disclosure, are for use in treating or preventing a viral infection, comprising administering an effective amount of the formulation to the subject, wherein, upon contacting an infectious agent, the formulation prevents transmission, infection, replication, survival or growth of the virus or bacteria in the subject. In some embodiments, the formulations of the disclosure, are for use in treating or preventing an infection transduced by a virus communicable by any means.

In some embodiments of the formulations of the disclosure for use in treating or preventing an infection transduced by a virus, the virus is communicated by contacting directly or indirectly an organism infected with the virus or contaminated by the virus.

Directly contacting an organism infected with the virus or contaminated by the virus include but are not limited to directly contacting any part, portion or surface of the organism or contacting any object infected with the virus or contaminated with the virus, surface or material that is or has been in contact with the organism infected with the virus or contaminated with the virus. The organism infected with the virus or contaminated by the virus can present one or more symptom(s) of the infection caused by the virus disease caused by the virus. The symptoms include but are not limited to: 1) fever or hyperthermia, 2) coughing, 3) sneezing, 4) chest congestion, 5) difficulty in breathing or shortness of breath, 6) frequent shivering, 7) muscle pain, 8) headache, 9) loss of taste or smell or both, 10) sore throat, 11) blood discharge in at least one of urine or fecal excretion, mucus, sputum, nasal discharge, urethral discharge, vaginal discharge or skin, 12) loss of weight, 13) loss of appetite, 14) occurrence of rashes or boils or sores or depigmentation or hyperpigmentation of skin, 15) loss of hair, and 14) loss of immunity to infections.

The organism infected with the virus or contaminated by the virus may not present any symptom(s) of the infection caused by the virus disease caused by the virus. The organism infected with the virus or contaminated by the virus is an asymptomatic carrier of the virus. The organism that is an asymptomatic carrier of the virus is a host or a harbor or a reservoir of the virus, wherein the virus survives and replicates in the organism.

The organism infected with the virus or contaminated by the virus can present one or more symptom(s) of the infection caused by the virus disease caused by the virus, is a host or a harbor or a reservoir of the virus, wherein the virus survives and replicates in the organism, and is dissipated or released from the organism into the environment surrounding the organism. The organism that is an asymptomatic carrier of the virus is a host or a harbor or a reservoir of the virus, wherein the virus survives and replicates in the organism, and dissipates from the organism in to the environment surrounding the organism. The virus dissipated or released from the organism infected with the virus or contaminated by the virus, into the environment surrounding the organism, can be transmitted or communicated to a subject that comes in contact with either the environment surrounding the organism. The virus dissipated or released from the organism infected with the virus or contaminated by the virus, into the environment surrounding the organism, can be transmitted or communicated to a subject that comes in direct or indirect contact with the virus in either the environment surrounding the organism or the organism itself.

In some embodiments of the formulations of the disclosure for use in treating or preventing an infection transduced by a virus, the subject contacts the virus by communication of the virus through a fluid media, the fluid media being extracted from, isolated from, secreted by or excreted by, the organism infected with the virus or contaminated by the virus. In some embodiments of the formulations of the disclosure for use in treating or preventing an infection transduced by a virus, the subject contacts the virus by communication of the virus through air exhaled or released by the organism infected with the virus or contaminated by the virus. In some embodiments of the formulations of the disclosure for use in treating or preventing an infection transduced by a virus, the fluid media being extracted from, isolated from, secreted by or excreted by, the organism infected with the virus or contaminated by the virus, comprises a bodily fluid or particulate thereof from the infected or contaminated organism. In some embodiments of the formulations of the disclosure for use in treating or preventing an infection transduced by a virus, the air exhaled or released by the organism infected with the virus or contaminated by the virus, comprises a bodily fluid or particulate thereof from the infected or contaminated organism. In some embodiments of the formulations of the disclosure for use in treating or preventing an infection transduced by a virus, the body fluid comprises exhaled or excreted droplets. In some embodiments of the formulations of the disclosure for use in treating or preventing an infection transduced by a virus, the body fluid comprises aerosolized droplets. In some embodiments of the formulations of the disclosure for use in treating or preventing an infection transduced by a virus, the droplets are between 10⁻⁴ to 10 μM in diameter or size. In some embodiments of the formulations of the disclosure for use in treating or preventing an infection transduced by a virus, the bodily fluid comprises sputum, saliva, blood, plasma, serum, lymph fluid, tears, sweat, urine or feces.

In some embodiments of the formulations of the disclosure for use in treating or preventing an infection transduced by a virus, the virus is communicated to the subject from across a physical distance of between 0.1 and 12 feet from the organism. In some embodiments of the formulations of the disclosure for use in treating or preventing an infection transduced by a virus, the virus is communicated to the subject from across a physical distance of 6 feet or less from the organism. In some embodiments of the formulations of the disclosure for use in treating or preventing an infection transduced by a virus, the virus is communicated to the subject from across a physical distance of less than 3 feet from the organism. In some embodiments of the formulations of the disclosure for use in treating or preventing an infection transduced by a virus, the virus is communicated to the subject from across a physical distance of less than 1 feet from the organism.

In some embodiments of the formulations of the disclosure for use in treating or preventing an infection transduced by a virus, the virus survives, remains viable or retains an infectious activity on a surface for between 0.1 minute and 6 months. In some embodiments, the virus survives, remains viable or retains an infectious activity on a surface for between 0.1 minute and 5 minutes. In some embodiments of the formulations of the disclosure for use in treating or preventing an infection transduced by a virus, the virus survives, remains viable or retains an infectious activity on a surface for between 5 minute and 15 minutes. In some embodiments of the formulations of the disclosure for use in treating or preventing an infection transduced by a virus, the virus survives, remains viable or retains an infectious activity on a surface for between 15 minute and 30 minutes. In some embodiments of the formulations of the disclosure for use in treating or preventing an infection transduced by a virus, the virus survives, remains viable or retains an infectious activity on a surface for between 1 hours and 3 hours. In some embodiments, the virus survives, remains viable or retains an infectious activity on a surface for between 24 hours and 72 hours. In some embodiments of the formulations of the disclosure for use in treating or preventing an infection transduced by a virus, the virus survives, remains viable or retains an infectious activity on a surface for between 1 day and 5 days. In some embodiments of the formulations of the disclosure for use in treating or preventing an infection transduced by a virus, the virus survives, remains viable or retains an infectious activity on a surface for between 1 week and 3 weeks. In some embodiments of the formulations of the disclosure for use in treating or preventing an infection transduced by a virus, the virus survives, remains viable or retains an infectious activity on a surface for between 1 month and 6 months. In some embodiments of the formulations of the disclosure for use in treating or preventing an infection transduced by a virus, the virus is the exhaled or excreted droplets or the aerosolized droplets remains viable or retains an infectious activity when floating in air for between 1 hour and 3 hours. In some embodiments of the formulations of the disclosure for use in treating or preventing an infection transduced by a virus, the virus in the exhaled or excreted droplets or the aerosolized droplets remains viable or retains an infectious activity when floating in air for between 1 hour and 6 hours. In some embodiments of the formulations of the disclosure for use in treating or preventing an infection transduced by a virus, the virus in the exhaled or excreted droplets or the aerosolized droplets remains viable or retains an infectious activity when floating in air for up to 1 hour. In some embodiments of the formulations of the disclosure for use in treating or preventing an infection transduced by a virus, the virus in exhaled, excreted or aerosolized droplets of size >5 μM remain suspended in air for longer time than virus in exhaled, excreted or aerosolized droplets of size <5 μM. In some embodiments of the formulations of the disclosure for use in treating or preventing an infection transduced by a virus, the bodily fluid or particulate thereof from the infected or contaminated organism, comprises droplets of varying sizes, e.g. >5 μM and <5 μM. In some embodiments of the formulations of the disclosure for use in treating or preventing an infection transduced by a virus, the survival of the virus in the exhaled or excreted droplets or the aerosolized droplets depends on the parameters of the air including but not limited to air temperature, air humidity, air pressure, air velocity, solar intensity or a combination thereof.

In some embodiments of the formulations of the disclosure for use in treating or preventing an infection transduced by a virus, the virus remains viable or retains an infectious activity when deposited on a surface. In some embodiments of the formulations of the disclosure for use in treating or preventing an infection transduced by a virus, the virus remains viable or retains an infectious activity on a surface, the surface comprises a biological surface. In some embodiments of the formulations of the disclosure for use in treating or preventing an infection transduced by a virus, the surface comprises one or more of a plant, a tree, a crop or a component thereof, skin, hair and nails. In some embodiments of the formulations of the disclosure for use in treating or preventing an infection transduced by a virus, the surface comprises one or more of silk, cotton, cellulose, cork, wool, wood, cardboard, latex, rubber and paper. In some embodiments of the formulations of the disclosure for use in treating or preventing an infection transduced by a virus, the surface does not comprise a biological surface. In some embodiments of the formulations of the disclosure for use in treating or preventing an infection transduced by a virus, the surface comprises an organic surface.

In some embodiments of the formulations of the disclosure for use in treating or preventing an infection transduced by a virus, the surface comprises one or more of carbon fiber, a plastic and synthetic fiber. In some embodiments of the formulations of the disclosure for use in treating or preventing an infection transduced by a virus, the surface comprises an inorganic surface. In some embodiments of the formulations of the disclosure for use in treating or preventing an infection transduced by a virus, the surface comprises one or more of a metal, silicone and glass. In some embodiments of the formulations of the disclosure for use in treating or preventing an infection transduced by a virus, the virus remains viable or retains an infectious activity when deposited on a surface, the surface being clothing, apparel, accessory, ornament, furniture, tool or food item that has been contacted direct or indirect contact with the organism that is infected by the virus or contaminated by the virus.

In some embodiments of the formulations of the disclosure for use in treating or preventing an infection transduced by a virus, the virus does not remain viable or retains an infectious activity when deposited on a surface if the surface is contacted with a disinfectant solution. In some embodiments of the formulations of the disclosure for use in treating or preventing an infection transduced by a virus, the disinfectant is any one of a 70% ethanol solution, a bleaching solution or any commercially available disinfectant solution or a combination thereof. In some embodiments of the formulations of the disclosure for use in treating or preventing an infection transduced by a virus, the virus does not remain viable or retains an infectious activity when deposited on a surface if the surface is contacted with ultraviolet (UV) light. In some embodiments of the formulations of the disclosure for use in treating or preventing an infection transduced by a virus, the subject can be prevented from contacting the virus on a surface or from the environment surrounding an organism infected with a virus or contaminated by a virus, by creating a barrier between the subject and the surface or the organism. In some embodiments of the formulations of the disclosure for use in treating or preventing an infection transduced by a virus, the barrier is a disposable National Institute of Occupational Safety and Health (NIOSH) approved N-95 filtering face piece respirator, a ANSI/AAMI PB70 highest barrier level gown, surgical gloves, or any personal protective equipment (PPE).

In some embodiments of the formulations of the disclosure for use in treating or preventing an infection transduced by a virus, the organism infected with the virus or contaminated with the virus is a human. In some embodiments of the formulations of the disclosure for use in treating or preventing an infection transduced by a virus, the organism infected with the virus or contaminated with the virus is not a human. In some embodiments of the formulations of the disclosure for use in treating or preventing an infection transduced by a virus, the organism is a mammalian, an avian, a reptilian, an amphibian, a crustacean, an artropod or a chordata organism. In some embodiments of the formulations of the disclosure for use in treating or preventing an infection transduced by a virus, the virus is a zoonotic virus. In some embodiments of the formulations of the disclosure for use in treating or preventing an infection transduced by a virus, contacting comprises consumption or handling of the organism by the subject. In some embodiments, the organism is a domesticated animal. In some embodiments of the formulations of the disclosure for use in treating or preventing an infection transduced by a virus, the domesticated animal is a pet or an ornamental animal. In some embodiments of the formulations of the disclosure for use in treating or preventing an infection transduced by a virus, the pet or ornamental animal is a dog, a cat, a bird, a reptile, a rodent including but not limited to a mouse, a rat, a rabbit, a hare and a hamster. In some embodiments of the formulations of the disclosure for use in treating or preventing an infection transduced by a virus, the organism is a live-stock animal. In some embodiments of the formulations of the disclosure for use in treating or preventing an infection transduced by a virus, the organism is a dog, a cat, a hamster, a rodent, a poultry including but not limited to hen, duck, goose, turkey and quail, a cattle including but not limited to cow, sheep and goat, a horse, a donkey, a mule, a llama, a camel, pig. In some embodiments, the organism is an aquatic animal including but not limited to a fish, a whale, a squid, an octopus, a crustacean, a reptile, or an amphibian.

In some embodiments of the formulations of the disclosure for use in treating or preventing an infection transduced by a virus, the organism is a wild animal. In some embodiments, the wild animal is a bat, a cat including but not limited to a lion, a tiger, a panther or a jaguar, a fox, a wolf, a coyote, a civet, a snake, a lizard, an amphibian, a rodent, a non-human primate or an ape species.

In some embodiments of the formulations of the disclosure for use in treating or preventing an infection transduced by a virus, the contacting occurs within a distance of 5 miles or less of: i) a human dwelling, ii) a laboratory or a research facility, ii) a market, store, or retail location, iv) a zoo, game reserve, wildlife reserve, land managed for wildlife protection or wildlife sanctuary, v) a farm, a field, or an agricultural location, vi) a hotel, a lodge, a resort or a site for an ecotourism activity, vii) a source of water, a well or a barrel maintained for drinking water, a stream, a river, a lake and an ocean, and/or viii) an airplane, a ship, a boat, a bus, a train, a car, a truck, or any other means of public, commercial or personal transportation. In some embodiments of the formulations of the disclosure for use in treating or preventing an infection transduced by a virus, the communication of the virus by the subject involves direct and indirect contact as follows i) wild animals in and around human dwellings, ii) wild animals hunted, iii) wild animals consumed, iv) wild animals kept as pets, v) wild animals housed in laboratories, vi) wild animals sold in markets, vii) wild animals kept in zoos and sanctuaries, viii) wild animal exposure during agricultural activities, ix) wild animal exposure during ecotourism activities, x) wild animal exposure during wildlife management activities in protected areas, xi) virus exposure in laboratory settings (lab pathogen), and xii) virus exposure via contaminated water. In some embodiments, contacting the organism is intentional. In some embodiments, the organism is personal property, state property, communal property, a hunting target, a food source, a research subject, a pet, a native species, an invasive species, a prey of any one of the foregoing or a predator of any one of the foregoing. In some embodiments, contacting the organism is unintentional. In some embodiments, the organism is a native species, an invasive species or a predator of the subject.

In some embodiments of the formulations of the disclosure for use in treating or preventing an infection transduced by a virus, the virus frequently mutates resulting in generation of strains or serotypes of the virus with altered genomes and surface antigens. In some embodiments of the formulations of the disclosure for use in treating or preventing an infection transduced by a virus, the virus frequently mutates resulting in generation of strains or serotypes of the virus that are not recognized by the immune system of the subject. In some embodiments of the formulations of the disclosure for use in treating or preventing an infection transduced by a virus. In some embodiments, the virus frequently mutates resulting in generation of strains or serotypes of the virus that are resistant to drugs and treatments, e.g., anti-retrovirals, antibodies and vaccines.

In some embodiments of the formulations of the disclosure for use in treating or preventing an infection transduced by a virus, the mutation rate of the genome of the virus is between 10⁻⁴ and 10⁻⁸ mutations per nucleotide per replication cycle. In some embodiments of the formulations of the disclosure for use in treating or preventing an infection transduced by a virus, the mutation rate of the genome of the virus is between 10⁻² and 10⁻⁵ nucleotide substitution per site per year. In some embodiments, the mutation rate of the genome of the virus is 0.80-2.38×10⁻³ nucleotide substitution per site per year. In some embodiments of the formulations of the disclosure for use in treating or preventing an infection transduced by a virus, the mutation rate of the genome of the virus is 1.16-3.30×10⁻³ nucleotide substitution per site per year for non-synonymous substitution and 1.67-4.67×10⁻³ nucleotide substitution per site per year for synonymous substitution.

In some embodiments of the formulations of the disclosure for use in treating or preventing an infection transduced by a virus, the RNA viruses mutate faster than DNA viruses.

In some embodiments of the formulations of the disclosure for use in treating or preventing an infection transduced by a virus, the mutation rate of the genome of the virus depends on the genetic composition or nucleic acid type of the virus and genome size of the virus. In some embodiments of the formulations of the disclosure for use in treating or preventing an infection transduced by a virus, the genome of a virus that is a RNA virus mutates at a higher rate than the genome of a virus that is a DNA virus. In some embodiments of the formulations of the disclosure for use in treating or preventing an infection transduced by a virus, the genome of a virus that is single stranded mutates at a higher rate than the genome of a virus that is double stranded. In some embodiments of the formulations of the disclosure for use in treating or preventing an infection transduced by a virus, the mutation rate of the genome of the virus correlates negatively with the genome size of the virus. In some embodiments of the formulations of the disclosure for use in treating or preventing an infection transduced by a virus, the mutation rates of the genome of the virus is modulated at different levels, including polymerase fidelity, sequence context, template secondary structure, cellular microenvironment, replication mechanisms, proofreading, and access to post-replicative repair. In some embodiments, the mutation rate of the genome of the virus can be influenced by the by virus-encoding diversity-generating elements, as well as by host-encoded cytidine/adenine deaminases.

In some embodiments of the formulations of the disclosure for use in treating or preventing an infection transduced by a virus, the genome of the virus has a medium to high mutation rate, medium mutation rate being >10⁻⁸ mutations per nucleotide per replication cycle and high mutation rate being >10⁻⁶ mutations per nucleotide per replication cycle.

In some embodiments of the formulations of the disclosure for use in treating or preventing an infection transduced by a virus, the genome of the virus undergoes high mutation rates and frequent genetic reassortment, resulting in frequent antigenic drift. Antigenic drift is a mechanism for variation by viruses that involves the accumulation of mutations, e.g. point mutations or deletion mutations) within the antibody-binding sites so that the resulting viruses cannot be inhibited well by antibodies against previous strains making it easier for them to spread throughout a partially immune population. Antigenic shift results in different strains of the same virus. In some embodiments of the formulations of the disclosure for use in treating or preventing an infection transduced by a virus, the antigenic drift results in alteration in amino acid sequence and structure of viral proteins. In some embodiments of the formulations of the disclosure for use in treating or preventing an infection transduced by a virus, the antigenic drift results in alteration in the amino acid sequence and structure of viral surface proteins. In some embodiments of the formulations of the disclosure for use in treating or preventing an infection transduced by a virus, the antigenic drift results in alteration in viral glycoprotein proteins Hemagglutinin (H or HA), Neuraminidase (N or NA), or both. In some embodiments of the formulations of the disclosure for use in treating or preventing an infection transduced by a virus, the altered viral glycoprotein proteins Hemagglutinin results in 18 Hemagglutinin variants, H1-H18. In some embodiments of the formulations of the disclosure for use in treating or preventing an infection transduced by a virus, the altered viral glycoprotein proteins Neuraminidase results in 11 Neuraminidase variants, N1-H11. In some embodiments of the formulations of the disclosure for use in treating or preventing an infection transduced by a virus, the antigenic drift results in alteration in viral glycoprotein proteins Hemagglutinin (H or HA), Neuraminidase (N or NA), or both, resulting in viral strains comprising a combination of any one of the Hemagglutinin variants H1-18 and any one of the Neuraminidase variants N1-11.

In some embodiments of the formulations for use in treating or preventing an infection, a genomic sequence of the virus undergoes reassortment. Reassortment is the process by which viruses with a segmented genome, like Influenza virus, swap gene segments. This genetic exchange is possible due to the segmented nature of the viral genome and occurs when two differing viruses e.g. Influenza viruses co-infect a cell. The viral diversity generated through reassortment is vast and plays an important role in the evolution of viruses like Influenza viruses. For example, if two different influenza virus infect a cell, the individual RNA segments of each Influenza virus enter the nucleus. RNAs of both viruses are copied in the nucleus and exported to the cytoplasm, and then are incorporated into new virus particles which bud from the cell. When new virus particles are assembled at the plasma membrane, each of the RNA segments may originate from either infecting virus. Viral progeny that inherit RNAs from both infecting parent viruses are called reassortants.

In some embodiments of the formulations for use in treating or preventing an infection, the genomic sequence of the virus mutates at a rate higher than 10⁻⁶ mutations per nucleotide of the genome per replication cycle.

In some embodiments of the formulations for use in treating or preventing an infection, the genomic sequence of the virus undergoes reassortment at a frequency of 6% to 20%. In some embodiments of the formulations for use in treating or preventing an infection, the genomic sequence of the virus undergoes reassortment at a frequency of 10% to 20%. In some embodiments of the formulations for use in treating or preventing an infection, the genomic sequence of the virus undergoes reassortment more frequently than 20%. In some embodiments of the formulations for use in treating or preventing an infection, the genomic sequence of the virus undergoes reassortment more frequently than 40%.

The time before the symptoms of a viral infection appear is called the incubation period. During this time, viral genomes are replicating and the host is responding, producing cytokines such as interferon that can have global effects, leading to the classical symptoms of an acute infection (e.g., fever, malaise, aches, pains, and nausea). These symptoms are called the prodrome, to distinguish them from those characteristic of infection (e.g. paralysis for poliovirus, hemorrhagic fever for Ebolaviruses, rash for measles virus). Whether or not an infected person is contagious (i.e. is shedding virus) during the incubation period depends on the virus. In some embodiments, the subject once infected or transmitted with the virus is contagious (i.e. shedding virus) during the incubation period. In some embodiments, the subject once infected or transmitted with the virus is not contagious (i.e. shedding virus) during the incubation period.

In some embodiments, the virus has an incubation period of 1 day to more than 1 year. In some embodiments, the virus has an incubation period of 1 week to more than 4 weeks. In some embodiments, the virus has an incubation period of 1 week to 4 weeks. In some embodiments, the virus has an incubation period of 1 to 21 days. In some embodiments, the virus has an incubation period of 1 to 15 days. In some embodiments, the virus has an incubation period of 0.5 to 15 days. In some embodiments, the virus has an incubation period of 0.5 to 1.5 days. In some embodiments, the virus has an incubation period of 5 to 15 days. The basic reproduction number or reproduction number (R0), also called the basic reproduction ratio or rate or the basic reproductive rate, is a metric used to describe the contagiousness or transmissibility of infectious agents, including a virus. R0 provides some information regarding the speed at which a disease is capable of spreading in a specific population. R0 is the number of secondary cases of infection that would result from a case in a specific population. An R0 for an infectious disease event is generally reported as a single numeric value or low-high range, and the interpretation is that a disease outbreak is expected to continue if R0 has a value >1, the disease will be stable if R0 has a value=1, and the disease will end if R0 is <1. The magnitude of the R0 value for a disease event can be used to determine the potential size of an outbreak or epidemic often is based on, and to estimate the proportion of the population that must be vaccinated to eliminate an infection from that population. In some embodiments of the formulations of the disclosure, the virus has a reproduction number (R0) between 0.9 and 18. In some embodiments, the virus has a R0 between 12 and 18. In some embodiments, the virus has a R0 between 1.5 and 2.5. In some embodiments, the virus has a R0 between 1.5 and 3.5. In some embodiments, the virus has a R0 between 2 to 5. In some embodiments, the virus has a R0 between 5 and 6.

In some embodiments of the formulations for use in treating or preventing an infection, the virus is an RNA virus.

In some embodiments of the formulations for use in treating or preventing an infection, the RNA virus is a positive-strand RNA virus. In some embodiments of the formulations for use in treating or preventing an infection, the positive-strand RNA virus belongs to the family of Picornaviridae, Astroviridae, Caliciviridae, Hepeviridae Flaviviridae, Togaviridae, Arteriviridae, or Coronaviridae.

In some embodiments of the formulations for use in treating or preventing an infection, the positive-strand RNA virus belongs to the family of Coronaviridae. In some embodiments of the formulations for use in treating or preventing an infection, the Coronaviridae virus is Bat coronavirus CDPHE15, Bat coronavirus HKU10, Rhinolophus ferrumequinum alphacoronavirus HuB-2013, Human coronavirus 229E, Lucheng Rn rat coronavirus, Ferret coronavirus, Mink coronavirus 1, Miniopterus bat coronavirus 1, Miniopterus bat coronavirus HKU8, Myotis ricketti alphacoronavirus Sax-2011, Nyctalus velutinus alphacoronavirus SC-2013, Porcine epidemic diarrhea virus, Scotophilus bat coronavirus 512, Rhinolophus bat coronavirus HKU2, Human coronavirus NL63, NL63-related bat coronavirus strain BtKYNL63-9b, Human coronavirus OC43, China Rattus coronavirus HKU24, Human coronavirus HKU1, Murine coronavirus-type species, Bat Hp-betacoronavirus Zhejiang2013, Hedgehog coronavirus 1, Middle East respiratory syndrome-related coronavirus (MERS-CoV), Pipistrellus bat coronavirus HKU5, Tylonycteris bat coronavirus HKU4, Rousettus bat coronavirus GCCDC1, Rousettus bat coronavirus HKU9, Severe acute respiratory syndrome-related coronavirus, Wigeon coronavirus HKU20, Bulbul coronavirus HKU11-type species, Porcine coronavirus HKU15, Munia coronavirus HKU13, White-eye coronavirus HKU16, Night heron coronavirus HKU19, Common moorhen coronavirus HKU21, Beluga whale coronavirus SW1 or Avian coronavirus-type species.

Coronaviridae virus or coronavirus is a large and a diverse group of positively-stranded RNA viruses with a nucleic acid size of 25,000 to 33,000 nucleotides. Coronavirus are known to cause a variety of pathological conditions in both humans and non-human animals. Coronavirus is composed of an envelope and a helical nucleocapsid with club-shaped surface projections that provide “attachment to cells, hemagglutination, and membrane fusion. Coronavirinae are divided into four genera: alpha-, beta-, gamma-, and delta-coronavirus.

In some embodiments of the formulations for use in treating or preventing an infection, the Coronaviridae virus is an alphacoronaviridae virus. In some embodiments of the formulations for use in treating or preventing an infection, the alpha-coronaviridae virus is Bat coronavirus HKU10, Human coronavirus 229E, Human coronavirus NL63, Miniopterus bat coronavirus 1, Miniopterus bat coronavirus HKU8, Mink coronavirus 1, Porcine epidemic diarrhoea virus, Rhinolophus bat coronavirus HKU2, Scotophilus bat coronavirus 512. In some embodiments of the formulations for use in treating or preventing an infection, the Coronaviridae virus is a beta-coronaviridae virus. In some embodiments of the formulations for use in treating or preventing an infection, the beta-coronaviridae virus is Betacoronavirus 1, Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus, Tylonycteris bat coronavirus HKU4. In some embodiments of the formulations for use in treating or preventing an infection, the Coronaviridae virus is a delta-coronaviridae virus. In some embodiments of the formulations for use in treating or preventing an infection, the delta-coronaviridae virus is Bulbul coronavirus HKU11, Common moorhen coronavirus HKU21, Coronavirus HKU15, Munia coronavirus HKU13, Night heron coronavirus HKU19, Thrush coronavirus HKU12, White-eye coronavirus HKU16, Wigeon coronavirus HKU20.

In some embodiments of the formulations for use in treating or preventing an infection, the Coronaviridae virus is a delta-coronaviridae virus. In some embodiments of the formulations for use in treating or preventing an infection, the delta-coronaviridae virus is Avian coronavirus or Beluga whale coronavirus SW1.

In some embodiments of the formulations for use in treating or preventing an infection, the Coronaviridae virus is Middle East respiratory syndrome-related coronavirus (MERS-CoV), Severe acute respiratory syndrome coronavirus (SARS-CoV) or Severe acute respiratory syndrome coronavirus 2 (SARS-CoV-2).

In some embodiments of the formulations for use in treating or preventing an infection, the Coronaviridae virus is Severe acute respiratory syndrome coronavirus 2 (SARS-CoV-2). In some embodiments of the formulations for use in treating or preventing an infection, the Severe acute respiratory syndrome coronavirus 2 (SARS-CoV-2) is the causative pathogenic agent for the coronavirus disease 2019 (COVID-19).

In some embodiments of the formulations for use in treating or preventing an infection, the RNA virus is a negative-strand RNA virus. In some embodiments of the formulations for use in treating or preventing an infection, the negative-strand RNA virus belongs to the family of Orthomyxoviridae, Paramyxoviridae, Rhabdoviridiae, Filoviruses, or Arenaviruses.

In some embodiments of the formulations for use in treating or preventing an infection, the negative-strand virus is Influenza virus, Sendai virus, Human parainfluenza virus 1 (hPIV1), Simian virus 5 (SV5, PIV5), Mumps virus, Newcastle disease virus (NDV), Measles virus, Rinderpest virus, Respiratory syncytial virus (RSV), Vesicular stomatitis virus (VSV), Rabies virus, Ebola virus, Marburg virus, Lymphocytic choriomeningitis virus (LCMV), Junin virus, or Lassa fever virus.

In some embodiments of the formulations for use in treating or preventing an infection, the negative-strand virus is Influenza virus. In some embodiments of the formulations for use in treating or preventing an infection, the Influenza virus is an Influenza virus Type A, B, C or D.

In some embodiments of the formulations for use in treating or preventing an infection, the Influenza virus is an Influenza virus Type A. In some embodiments of the formulations for use in treating or preventing an infection, the Influenza virus Type A is an Influenza virus Type A (H3N2). In some embodiments of the formulations for use in treating or preventing an infection, the Influenza virus Type A is an Influenza virus Type A (H1N1).

In some embodiments of the formulations for use in treating or preventing an infection, the Influenza virus is an Influenza virus Type B. In some embodiments of the formulations for use in treating or preventing an infection, the Influenza virus Type B is an Influenza virus Type B (Victoria). In some embodiments of the formulations for use in treating or preventing an infection, the Influenza virus Type B is an Influenza virus Type B (Yamagata).

In some embodiments of the formulations for use in treating or preventing an infection, the virus is a retrovirus. In some embodiments of the formulations for use in treating or preventing an infection, the retrovirus is a Lentivirus, Alpharetrovirus, Betaretrovirus, Deltaretrovirus, Gammaretrovirus or Epsilonretrovirus. In some embodiments of the formulations for use in treating or preventing an infection, the retrovirus is a Simian immunodeficiency virus (SIV), Human immunodeficiency virus-1 (HIV-1), HIV-2, Feline immunodeficiency virus (Hy), Equine infectious anemia virus (EIAV), Mouse mammary tumor-like virus (MMTV), Mason-Pfizer monkey virus (MPMV), Respiratory syncytial virus RSV, bovine leukemia virus (BLV), Human T-cell leukemia virus-1 (HTLV-1), HTLV-2, Murine leukemia virus (MuLV), Gibbon ape leukemia virus (GALV).

In some embodiments of the formulations for use in treating or preventing an infection, the virus is a DNA virus. In some embodiments of the formulations for use in treating or preventing an infection, DNA virus is Adenovirus, infectious canine hepatitis virus, Papillomavirus, polyomaviridae, simian vacuolating virus, Parvovirus B19, canine parvovirus, Herpes simplex virus, varicella-zoster virus, cytomegalovirus, Epstein-Barr virus, Smallpox virus, cow pox virus, sheep pox virus, monkey pox virus, vaccinia virus, Torque teno virus.

### Bioavailability

In some disclosures of the formulations of the disclosure, the formulations are designed to facilitate and/or to control release of niclosamide from the formulation to increase systemic bioavailability of niclosamide.

With respect to Niclocide (trade name: YOMESAN®) (www.bayer.co.za/static/documents/MSDS/PIs/YOMESAN_EN_PI.pdf), from which the company and corresponding regulatory data states niclosamide is poorly absorbed and has no systemic therapeutic effects. Niclocide is provided as a dry tablet containing 500 mg niclosamide and the following excipients: maize starch, talcum, sodium lauryl sulphate, povidone, vanillin, magnesium stearate, and saccharin sodium. Niclocide was formerly approved for use as a treatment for tapeworms in adults and in children. The formulation of the tablet was designed to limit absorption by the human subject in order to maximize exposure of the tapeworm to the niclosamide contained in the tablet. Thus, the systemic administration of Niclocide and similar formulations demonstrating poor absorption, and therefore, no systemic therapeutic effects, provided a topical treatment to the gastrointestinal tract of the human subject.

In contrast to dry formations of niclosamide that were administered orally for the treatment of tapeworms, in which the systemic administration did not result in either absorption of the niclosamide or in a systemic therapeutic effect, the formulations of the disclosure may be systemically administered and the niclosamide systemically absorbed. In some embodiments, the formulations of the disclosure increase release of niclosamide by increasing the relative abundance of a dispersant when compared to other excipients in the formulation. In some embodiments, the formulations of the disclosure comprise at least 3% sodium lauryl sulfate (SLS) or at least about 3% SLS. In some embodiments, the formulations of the disclosure comprise 5% SLS or about 5% SLS. Alternatively, or in addition, formulations of the disclosure increase the surface to area ratio of the dry or solid formation of the disclosure by formatting the dry or solid composition as a particulate. In some embodiments, the particulate comprises a plurality of spheres or microspheres.

To improve the safety profile of the formulations of the disclosure when compared to, for example, Niclocide and similar formulations containing talc or talcum, formulations of the disclosure do not contain talc or talcum.

### Pharmaceutical Forms and Components

In some embodiments, the formulation of the disclosure is a pharmaceutical formulation.

In some embodiments, pharmaceutical formulations of the disclosure comprise a solid form. In some embodiments, the solid form of the disclosure may comprise one or more of a particulate, a granule, a powder, a capsule, a tablet, an encapsulated semi-solid, an encapsulated liquid, a non-biodegradable implant, a biodegradable implant (e.g. for releasing the Niclosamide or pharmaceutically acceptable salt thereof), a deformable matrix, a polymer matrix, and a film. In some embodiments, the solid form comprise one or more of a filler, an excipient, an anti-adherent, a coating, a coloring agent, a glidant, a preservative, a sorbent, a bulking agent, a lubricating agent, an anti-oxidant, a binding agent, a disintegration agent, and a buffering agent.

In some embodiments, the pharmaceutical formulation of the disclosure comprise a matrix. In some embodiments, the matrix of the pharmaceutical composition of the disclosure may comprise a material including, but not limited to, hydrophobic matrices (e.g., polyethylene, polyvinyl chloride, ethyl cellulose and acrylate polymers and their copolymers), lipid matrices, hydrophilic matrices, cellulose derivatives (e.g. Methylcellulose); hydroxyethylcellulose (e.g. hydroxypropylmethyl cellulose (HPMC)); sodium carboxymethylcellulose; non cellulose natural or semi synthetic polymers (e.g. agaragar); carob gum; alginates; molasses; polysaccharides of mannose and galactose, chitosan and modified starches; polymers of acrylic acid (e.g. Carbopol-934), and bio-degradable matrices and mineral matrices.

Excipients of the disclosure include, but are not limited to, inorganic chemicals (e.g., calcium phosphates, calcium carbonate, calcium sulfate, halites, metallic oxides), organic chemicals (e.g., carbohydrates, sugars, actual sugars, sugar alcohols, artificial sweeteners), starch (e.g., modified starch, dried starch, converted starch), cellulose (e.g., cellulose ethers, cellulose esters, CMC and croscarmellose sodium, microcrystalline cellulose), petrochemicals, glycols (e.g., polyethylene glycol and propylene glycol), povidones, mineral hydrocarbons (petrolatum, mineral waxes, and mineral oils), acrylic polymers, some petrochemical excipients, oleochemicals (e.g., fatty alcohols, mineral stearates, glycerin), some oleochemical excipients and proteins.

Binding agents of the disclosure include, but is not limited to, saccharides and their derivatives (e.g., disaccharides: sucrose, lactose), polysaccharides and their derivatives (e.g., starches, cellulose or modified cellulose such as microcrystalline cellulose and cellulose ethers such as hydroxypropyl cellulose (HPC)), sugar alcohols (e.g., xylitol, sorbitol or maltitol), protein gelatin, and synthetic polymers (e.g., polyvinylpyrrolidone (PVP), polyethylene glycol (PEG)).

Disintegration agents of the disclosure include, but is not limited to, crosslinked polymers (e.g., cross-linked polyvinylpyrrolidone (crospovidone), cross-linked sodium carboxymethyl cellulose (croscarmellose sodium) and modified starch sodium starch glycolate.

Fillers of the disclosure include, but is not limited to, plant cellulose, dibasic calcium phosphate, vegetable fats and oils, lactose, sucrose, glucose, mannitol, sorbitol, calcium carbonate, and magnesium stearate.

Preservatives of the disclosure include, but is not limited to, antioxidants (e.g. vitamin A, vitamin E, vitamin C, retinyl palmitate, and selenium), amino acids (cysteine and methionine), and citric acid, sodium citrate and synthetic preservatives including parabens (e.g., methyl paraben and propyl paraben).

Pharmaceutical formulations of the disclosure may comprise suitable carriers, buffers, excipients, and agents that are incorporated into formulations to provide improved transfer, delivery, tolerance, and the like. A multitude of appropriate formulations can be found in the formulary known to all pharmaceutical chemists: Remington's Pharmaceutical Sciences (15th Ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman WN “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

### Pharmaceutically Acceptable Carriers

Niclosamide or pharmaceutically acceptable salt thereof(s) (also referred to as “Niclosamide or pharmaceutically acceptable salt thereof compounds” or “compounds”) may be combined with a pharmaceutically-acceptable carrier to produce formulations or compositions suitable for therapeutic administration in vivo, in vitro or ex vivo. The term “pharmaceutically acceptable carrier” includes, but is not limited to, any and all dispersion media, coatings, isotonic and absorption delaying agents, and the like, compatible with pharmaceutical administration.

A formulation of the disclosure is formulated to be compatible with its intended route of administration. In some embodiments, a formulation of the disclosure comprises a pharmaceutically-acceptable carrier that is compatible with the intended route of administration of the formulation. In some embodiments, a formulation of the disclosure further comprise an additional compound or agent to render the formulation compatible with its intended route of administration. Examples of routes of administration include, but are not limited to, parenteral, e.g., intradermal, subcutaneous, transdermal (i.e., topical). Formulations or the pharmaceutically-acceptable carriers therein used for parenteral, intradermal, or subcutaneous application may include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The formulations or the pharmaceutically-acceptable carriers therein suitable for injectable use may include sterile aqueous solutions (where water soluble) or dispersions and sterile powders for the extemporaneous preparation of sterile injectable solutions or dispersion. In preferred embodiments, pharmaceutical formulations or the pharmaceutically-acceptable carriers therein are stable under the conditions of manufacture and storage and are preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid and the like. In many cases, it will be preferable to include isotonic agents, for example, sugars, polyalcohols such as manitol, sorbitol, sodium chloride in the formulation. Prolonged absorption of the injectable compositions can be brought about by including in the formulation an agent which delays absorption, for example, aluminum monostearate and gelatin.

Sterile injectable pharmaceutical formulations or the pharmaceutically-acceptable carriers therein can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, niclosamide or pharmaceutically acceptable salt thereof(s) are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Liposomal suspensions can also be used as pharmaceutically acceptable carriers.

Pharmaceutical formulations comprising niclosamide or pharmaceutically acceptable salt thereof(s) may comprise a dosage unit form for ease of administration and uniformity of dosage. Dosage unit form, as used throughout the disclosure, refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, tube, or dispenser together with instructions for administration.

### Therapeutically Effective Amounts

A therapeutically effective dose may be a therapeutically effective amount of the formulation of the disclosure, that when contacted with a sperm cell, is sufficient to treat a viral infection. A therapeutically effective dose may be a portion of a therapeutically effective amount. For example, a therapeutically effective dose may be half of the therapeutically effective amount provided in two separate administrations over a period of time, that in aggregate, provide the therapeutically effective amount of the formulation.

A therapeutically effective amount of the formulation of the disclosure relates generally to the amount needed to achieve a therapeutic objective. A “therapeutically effective amount” of the formulation of the disclosure is an amount of the formulation that when contacted with the subject or the virus, for a sufficient amount of time, decreases or reduces one or more of a viral load, a number (absolute or relative) of viral particles or infected cells, reduces a severity of a sign or symptom of the infection, and/or improves one or more aspects of a prognosis for the subject.

A “sufficient amount of time” of contacting the formulation of the disclosure with a semen sample relates generally to the amount needed to achieve a therapeutic objective. “Sufficient amount of time” of the formulation of the disclosure is an amount of time for which when the formulation, when contacted, with a subject or a virus, decreases or reduces one or more of a viral load, a number (absolute or relative) of viral particles or infected cells, reduces a severity of a sign or symptom of an infection, and/or improves one or more aspects of a prognosis for the subject.

A “therapeutically effective amount” or “an effective amount” of the formulation of the disclosure also relates the amount needed to achieve complete inhibition or reduction in a viral titer (e.g. of a coronavirus or a SARS-CoV-2 virus) in a cell treated with the formulation of the disclosure, as compared to an untreated or control cell (vehicle treated cell). A “therapeutically effective amount” of the formulation of the disclosure also relates the amount needed to achieve inhibition or reduction in replication of a virus (e.g. of a coronavirus or a SARS-CoV-2 virus) in a cell treated with the formulation of the disclosure, as compared to an untreated or control cell (vehicle treated host cell). In some embodiments, a cell treated with a therapeutically effective amount of the formulation of the disclosure, upon infection with a virus (e.g. of a coronavirus or a SARS-CoV-2 virus), produces a viral titer of at least 50%, 60%, 70%, 80%, 90% or 100% lower than the viral titer produced by an untreated host cell or a control cell.

A “therapeutically effective amount” or “an effective amount” of the formulation of the disclosure is an amount of a niclosamide formulation sufficient to treat or prevent infection by a virus. In some embodiments, the subject receiving a “therapeutically effective amount” or “an effective amount” of the formulation of the disclosure is exposed to a virus (e.g. of a coronavirus or a SARS-CoV-2 virus) but is not infected by the virus (does not present signs or symptoms of infection). In some embodiments, the subject receiving a “therapeutically effective amount” or “an effective amount” of the formulation of the disclosure has been exposed to a virus or infected by a virus (e.g. of a coronavirus or a SARS-CoV-2 virus) but, following administration of the therapeutically effective amount of the formulation, demonstrates a reduction in a severity of a sign or symptom of the infection (including a reduction in a viral titre value from before and after administration of the formulation).

The subject can be a male subject or a female subject. The subject can be a person previously vaccinated with an anti-viral vaccine, e.g., COVID-19 vaccine. In some aspects, the subject is not previously vaccinated. In some aspects, the subject is less than 16 years old or less than 12 years old. In some aspects, the subject is less than 12, 11, 10, 9, 8, 7, 6, 5, 4, 3, 2, or 1 years old. In some aspects, the subject has already been infected with a virus. In some aspects, the subject has already been infected by a virus and recovered from the virus.

The “effective amount” of the formulation can be administered systemically, and preferably, orally, nasally, or intravenously.

### Niclosamide Capsules

Niclosamide is on the WHO's list of essential medicines and traditionally used to treat tapeworm infestations. ANA therapeutics demonstrated the efficacy of Niclosamide to treat SARS-CoV-2 (virus that causes COVID-19) from replicating and is planning to perform clinical trial to assess the efficacy of Niclosamide to treat COVID-19.

Described herein is the formulation development of Niclosamide capsules for clinical trials for COVID-19.

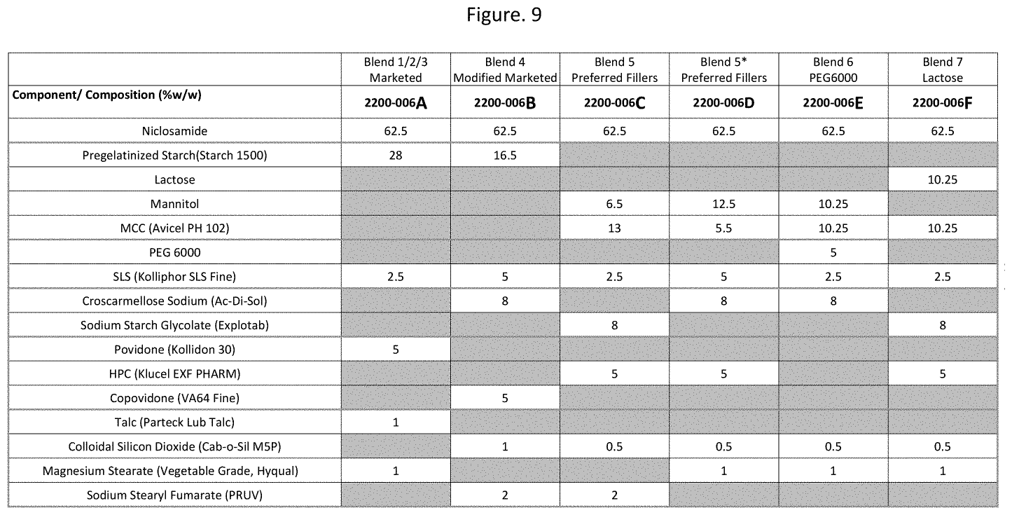

Prototype screening was conducted in parallel with excipient compatibility studies. Six (6) prototype formulations were prepared as found in FIG. 9. The physical mixtures of these prototypes were blended and compressed to form slugs at 12,000, 20,000 and 30,000 N on a manual tablet press. To determine the relative compressibility of the six different formulations, the hardness of slugs formed were tested using the tablet hardness tester.

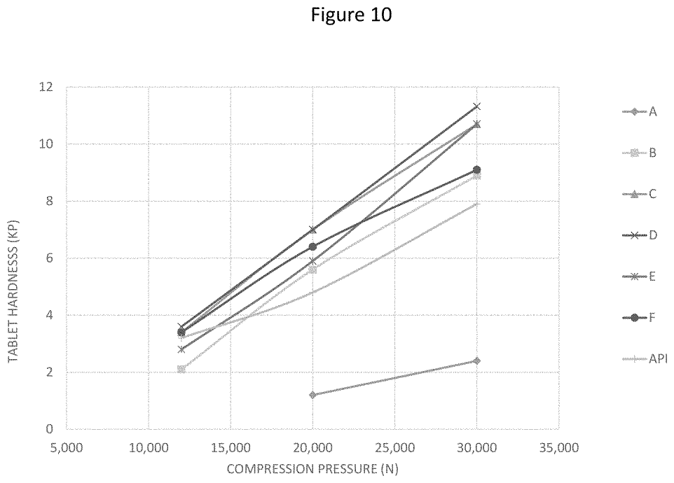

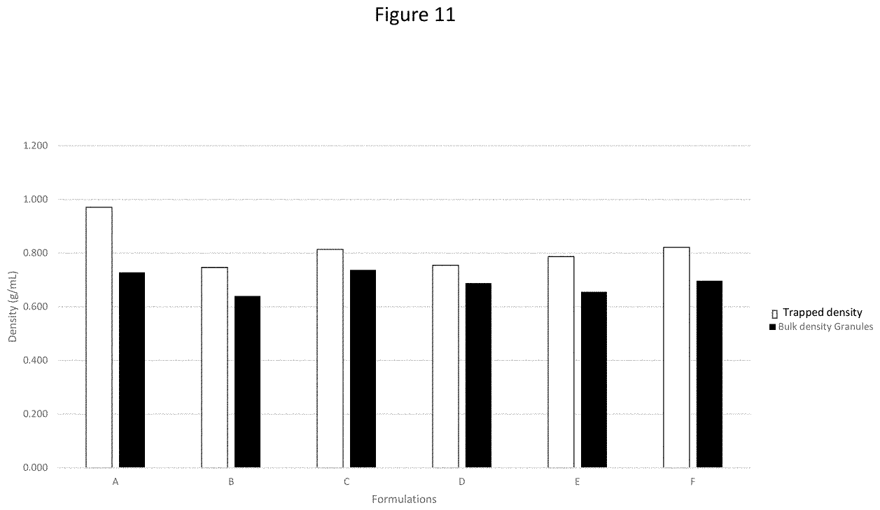

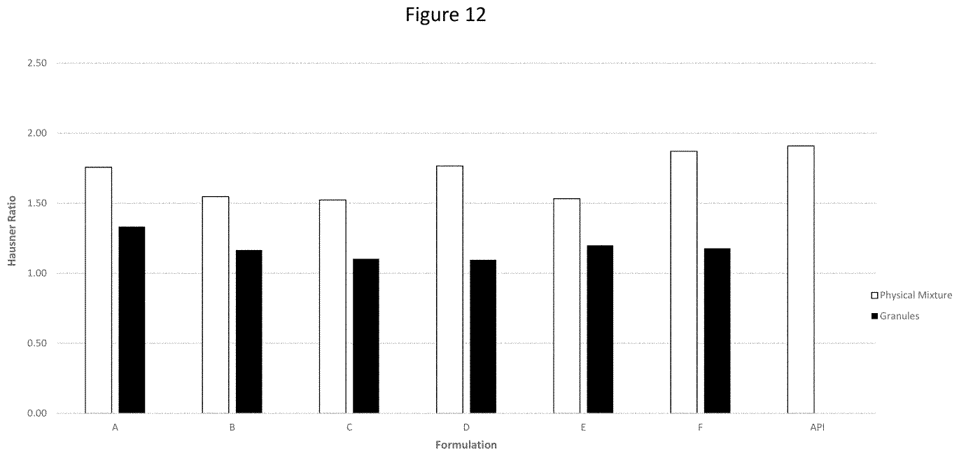

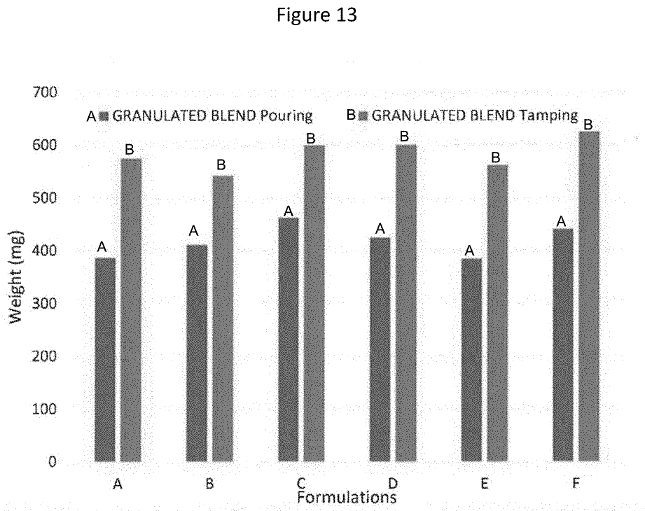

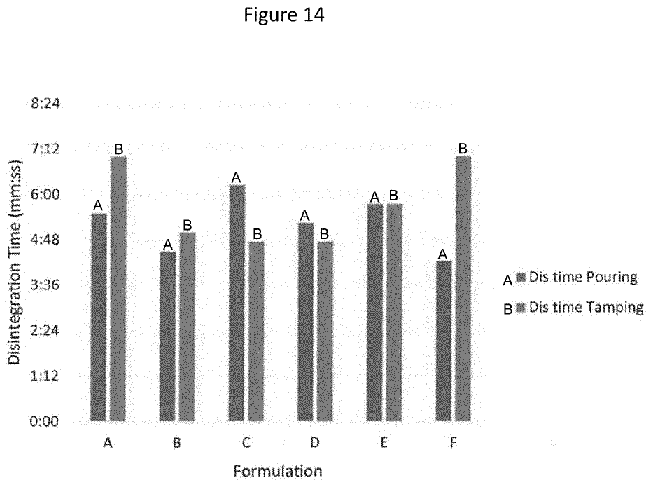

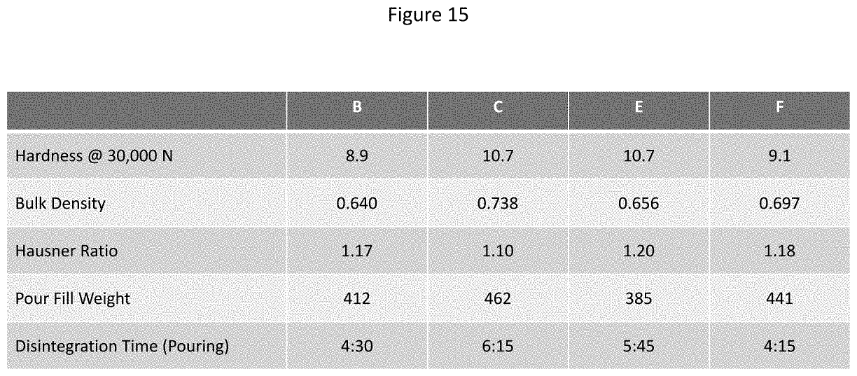

The formed slugs were then crushed using a mortal pestle and passed through #16 mesh sieve. The screened granules were evaluated for bulk/tapped density and pour/tamped filling into Size 0 capsule shells. Capsule disintegration was observed in a beaker containing potable water, and the capsule and rupture time was noted. The results of the screening are presented in FIGS. 10 to 14.

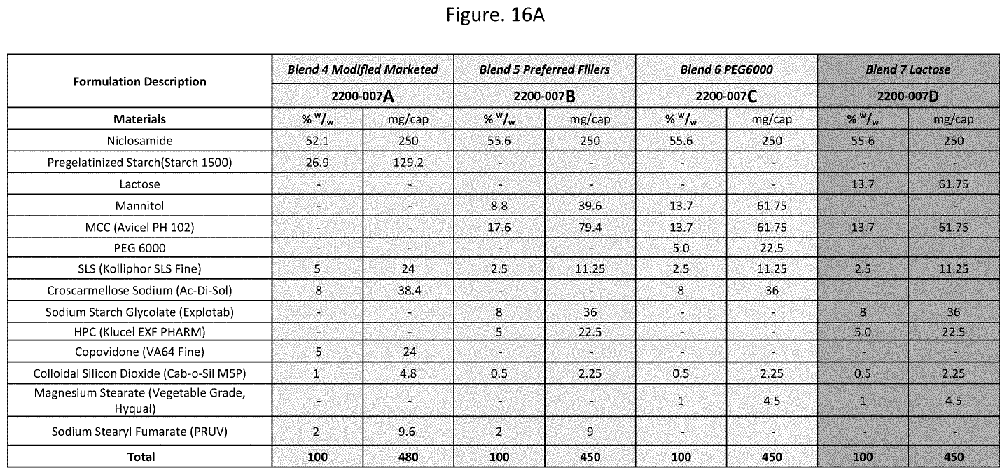

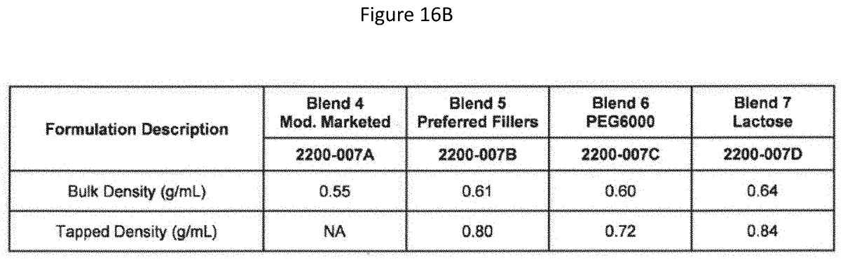

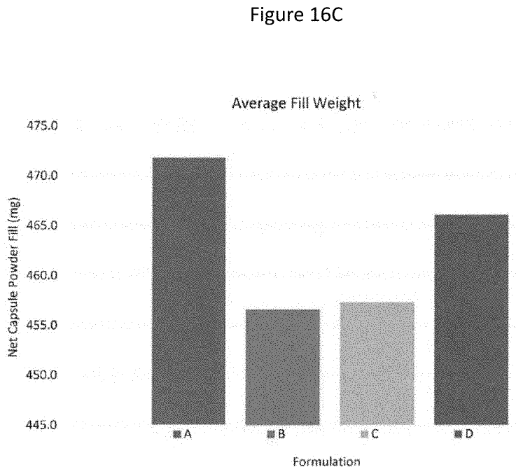

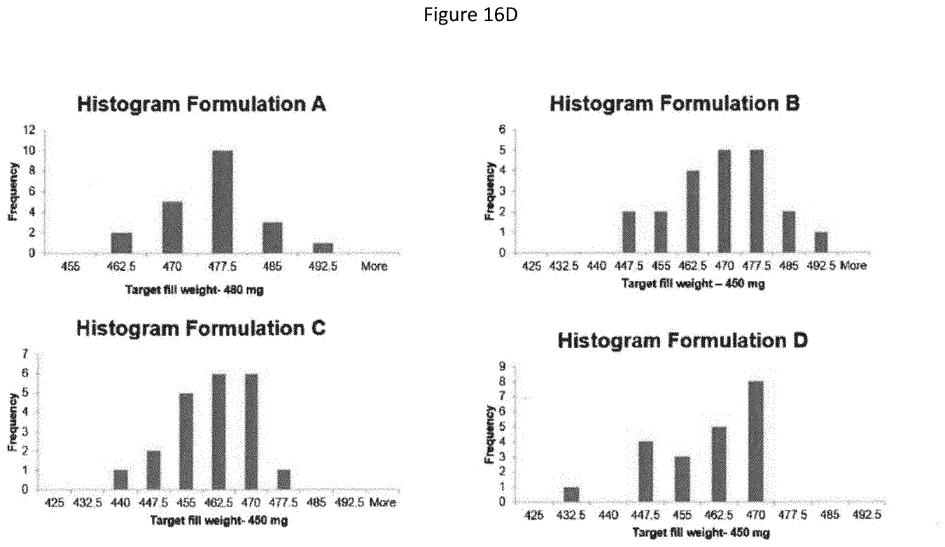

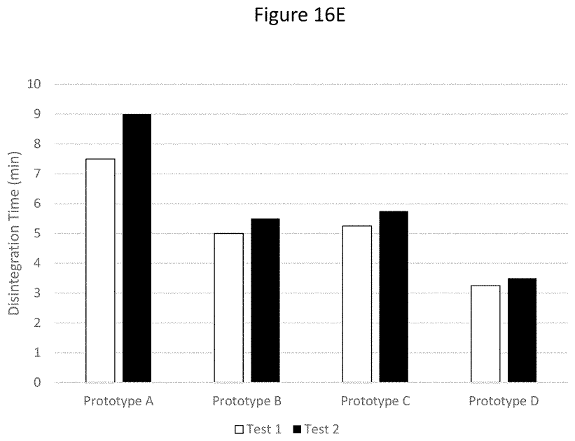

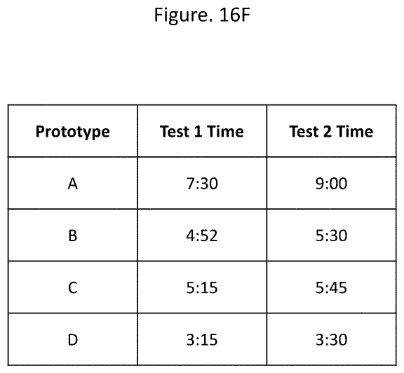

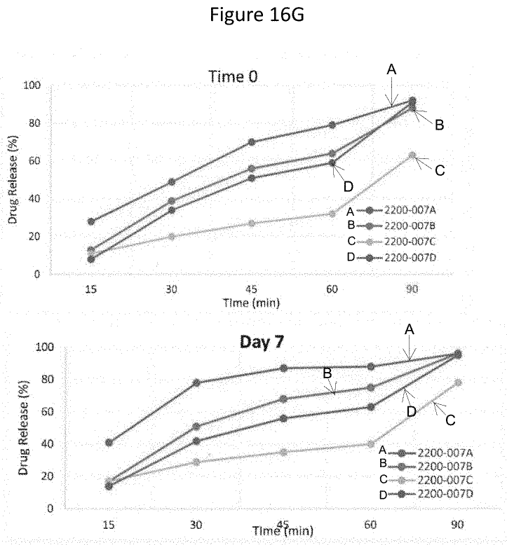

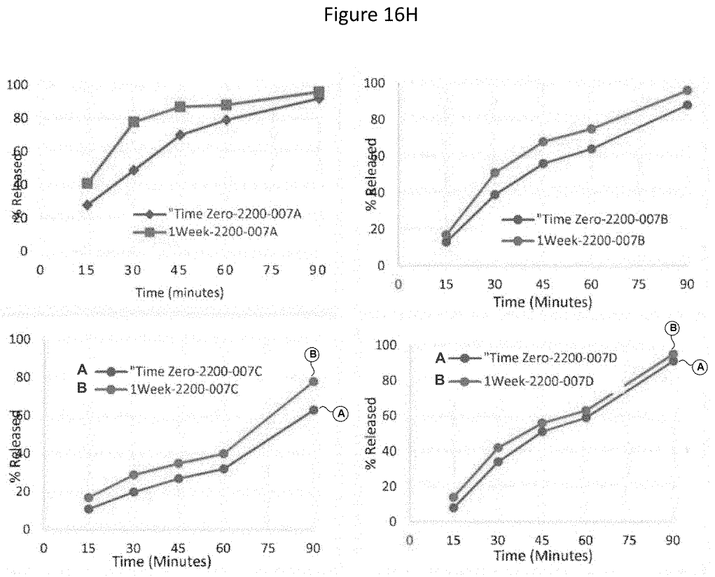

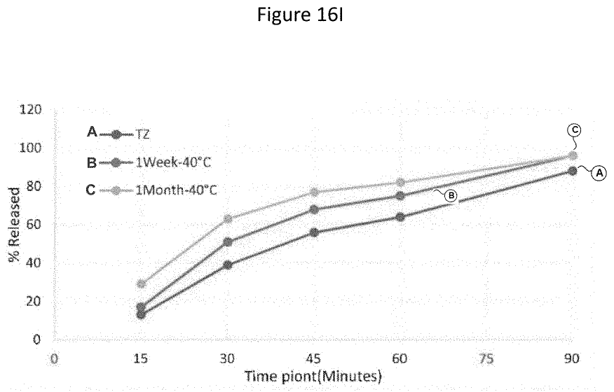

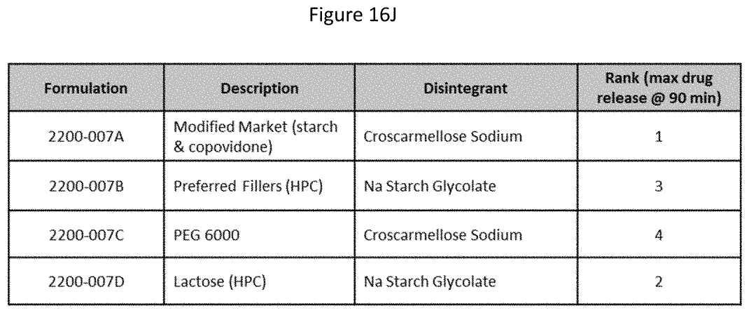

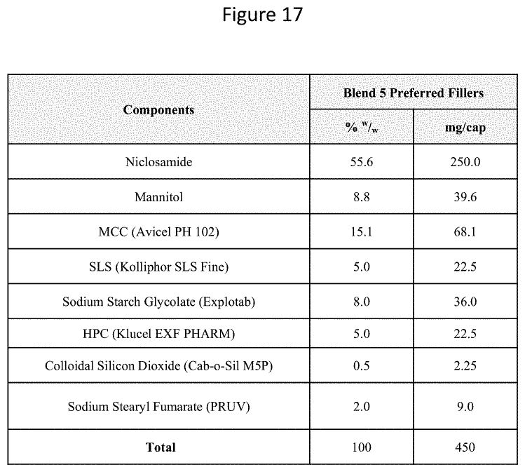

Four lab scale development batches were prepared to screen for a potential lead formulation to support clinical trial manufacture as found in FIG. 16A. The traceability between the blends identified in FIG. 9 and the development batches is made in the formulation description of FIG. 16A. The physical mixtures were blended and compressed at −15,000 N to form an acceptable slug of approximately 5.5 kp to ensure comparable granules particles and bulk/tapped density for all formulations. The slugs were then crushed by using mortar and pestle and passed through #16 mesh. The screened granules were filled into Size 0 capsules using Profill 100. The granules were analyzed for bulk/tapped density, PSD and pour/tamp filling in capsules. Final filled capsules were analyzed for weight variation, assay, related substances and dissolution. To evaluate chemical stability of a potential lead prototype, prototypes were placed on stability at 40″C/75% RH and analyzed for assay, related substances and drug release at 1, 4 and 12 weeks (FIGS. 16B-16I). The composition and physical and analytical testing results for these development batches are presented in subsequent sections.

Capsules from batch #2200-007 A, 2200-0078, 2200-007C and 2200-007D were analyzed for assay, related substances and drug release on day 0, 1 week and 4 weeks (FIGS. 16G-16I). The results for the tests are given below. All the prototype formulations showed assay values between 90-110% of label claim.

Drug release study was conducted by immersing Niclosamide capsules with sinker in pH 6.8 phosphate buffer containing 3.5% CTAB using USP Dissolution Apparatus I (paddle type). All four formulations show distinct drug release profile. Batch #2200-007C was not selected for further development as it had the least favorable dissolution profile. The Batch #2200-007 A formulation exhibit showed quickest and highest drug release on Day 0 and Day 7 compared to other three formulations. The drug release of Batch #2200-0078 tested for drug release at 1 month and found to be comparable for 3 time points (FIG. 16G and FIG. 16H).

Formulation (batch #2200-0078) containing MCC and mannitol with 5% w/w SLS was selected for further development. Although formulation B showed a slightly lower drug release than formulation A, slight modifications to the formulation including an increase in amount of SLS may improve drug release.

The proposed formulation to support the clinical trials is shown in Table 1. The schematic process flow chart is shown in Table 4.

TABLE 1
Proposed Formulation for CTM Batch (final formulation)

| | | |
|---|---|---|
| | Composition | |
| Description | (% w/w) | mg/Capsule |

Intra-Granular

| | | |
|---|---|---|
| Niclosamide | 55.6 | 250.0 |
| Mannitol | 8.8 | 39.6 |
| MCC Avicel PH-102 | 15.1 | 68.1 |
| SLS (Kolliphor SLS Fine) | 5.0 | 22.5 |
| Sodium Starch Glycolate (Portion I) | 5.0 | 22.5 |
| HPC (Klucel EXF) | 5.0 | 22.5 |
| Colloidal Silicon Dioxide (Cab-o-Sil M5P) | 0.3 | 1.4 |
| (Portion I) | | |
| Sodium Stearyl Fumarate (PRUV) (Portion I) | 1 | 4.5 |
| Total (Intra-Granular) | 95.8 | 431.1 |

Extra-Granular

| | | |
|---|---|---|
| Sodium Starch Glycolate (Portion II) | 3.0 | 13.5 |
| Colloidal Silicon Dioxide (Cab-o-Sil M5P) | 0.2 | 0.9 |
| (Portion II) | | |
| Sodium Stearyl Fumarate (PRUV) (Portion II) | 1 | 4.5 |
| Size 0 HPMC Capsules | N/A | 1 each |
| Total (Extra-Granular) | 4.2 | 18.9 |
| TOTAL (Capsule) | 100 | 450.0 |

A scale-up batch of 500 grams (Batch #2200-010) utilizing API supplied for R&D was manufactured on a Gerteis Mini-Pactor to determine the processability of the blend for roller compaction. The lead formulation, Formulation 8, was roller compacted by varying compaction force and roll gap, while maintaining other target process parameters as detailed in Table 2. The bulk density of granules at various process parameters is given in Table 3.

TABLE 2
Roller Compaction Parameters and Range in the Scale-up Batches
Gerteis Mini-Pactor Set-up Parameters

| | |
|---|---|
| Compaction force (kN/cm) | Varied |
| Gap width (mm) | Varied |
| Percent Draw in force (%) | 50 |
| Roller speed (rpm) | 2 |
| Tamp-to-feed auger ratio | 165 |
| Granulator speed (rpm) | 65 |
| Granulator angle, clockwise (°) | 250 |
| Granulator angle, counterclockwise (°) | 350 |
| Screen size (mm) | 1.0 mm |
| Roller type (Left) | Smooth |
| Roller type (Right) | Knurled |
| Granulator type | Star |
| Torque control | OFF |
| Gap Control | ON |
| PI Setting | Slow (1. 1000) |

TABLE 3
Granules Bulk Density During Roller Compaction Process

| | Compaction Force | Gap Width | Roll Speed | Bulk Density |
|---|---|---|---|---|
| Run | (kN/cm) | (mm) | (rpm) | (g/mL) |
| 1 | 10 | 1.0 | 11 | 0.583 |
| 2 | 6 | 1.0 | 1 | 0.568 |
| 3 | 11 | 1.0 | 1 | 0.629 |
| 4 | 11 | 2.0 | 1 | 0.596 |
| 5 | 12 | 2.0 | 1 | 0.616/0.59/0.63* |
| 6 (Target) | 12 | 1.0 | 1 | 0.626* |

*Bulk density was determined at 3 time-intervals during the sun
*Refers to bulk density of composite samples

The bulk density of granules from previous development batches which used manual slugging process was found to be 0.61 g/ml. As this bulk density was found to previously provide ample fill volume in a capsule and still required powder tamping, attempts were made to match this bulk density during roller compaction during the scale-up batch.

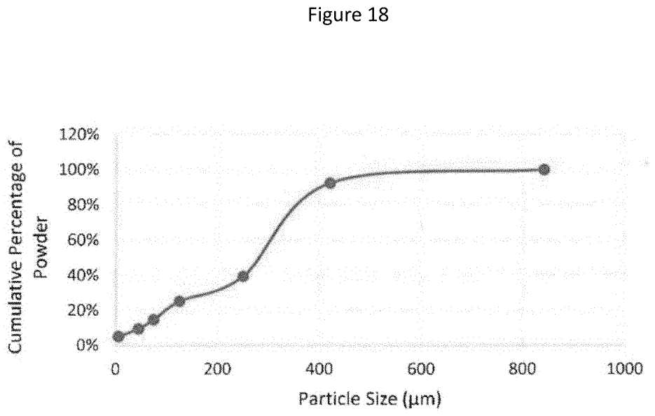

Roller compacted granules from the Target Roller Compaction Run 6 (about 0.61 g/ml bulk density) were then blended with extra granular excipients and filled into size 0 capsules using Profill. Although a capsule fill weight of 450 mg was targeted, the resulting average capsule fill weight was found to be about 470 mg approximately 5% greater than the original target with no tamping required. The particle size distribution of the final blend from this batch can be seen in FIG. 18.

The targeted density of roller compacted material would likely need to be reduced from 0.61 g/ml during CTM batch manufacture to achieve the target capsule fill weight of 450 mg. The estimated range bulk density range of 0.52-0.60 g/ml for roller compacted granules is expected to provide the targeted fill weight. Furthermore, the process parameters utilized during the roller compaction Target Run 6 are considered to be upper limit of the preferred operating space for future roller compaction processing if capsule fill volumes are to be maximized.

The detail process flow diagram for Niclosamide Capsules is shown in Table 4.

TABLE 4
Niclosamide Capsules Process CPP and CQA

| | Critical Process | Critical Quality |
|---|---|---|
| Process | Parameters/Controls | Attributes |
| Screening | Mesh size (20) | Particle Size Distribution |
| Blending | Blend Time (25 min) | Content Uniformity |
| | Blend Speed (fixed speed) | |
| Roller Compaction | Compaction Force (5 kN) | Bulk/Tapped Density, |
| | Roll Gap (2.0 mm) | Flow, |
| | Roll Speed (1 RPM) | Particle Size Distribution |
| Lubrication | Blend Time (5 min) | Content Uniformity |
| | Blend Speed (fixed speed) | |
| Encapsulation | Capsule Weight (450 mg) | Assay, |
| | | Content Uniformity, |
| | | Disintegration, |
| | | Dissolution |
| Primary | N/A | Full Count and |
| packaging and | | Full Label |
| Labelling | | Accountabliity |

Every document cited herein, including any cross referenced or related patent or application, is hereby incorporated herein by reference in its entirety unless expressly excluded or limited. The citation of any document is not an admission that it is prior art with respect to any invention disclosed or claimed herein or that it alone, or in any combination with any some reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the disclosure have been illustrated and described, various some changes and modifications can be made without departing from the spirit and scope of the disclosure. The scope of the appended claims includes all such changes and modifications that are within the scope of this disclosure.

### EXAMPLES

In order that the invention disclosed herein may be more efficiently understood, examples are provided below. It should be understood that these examples are for illustrative purposes only and are not to be construed as limiting the invention in any manner.

### Example 1

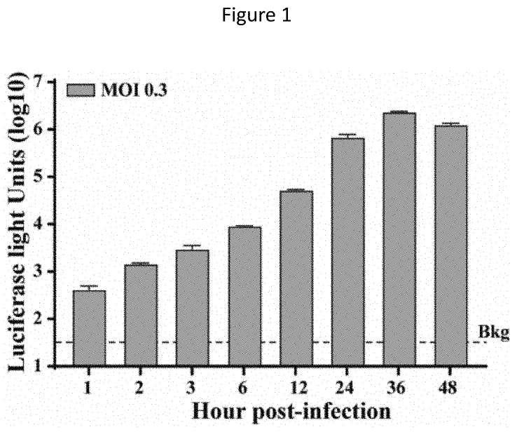

Antiviral assay: This reverse genetic system allows manipulation of the viral genome at any nucleotide position and engineering foreign into the virus (see, Xie et al., 2020). To facilitate high-throughput antiviral testing, a nano luciferase gene was engineered into the open-reading-frame 7 (ORF7) of the SARS-CoV-2 genome. When cells were infected with such reporter virus, the cells expressed luciferase that could be quantified to indicate viral replication. As shown in FIG. 1, increasing amounts of luciferase signals were detected in Vero E6 cells after infected with the reporter virus from 1 to 36 h post-infection. At 36 h post-infection, viral infection-induced cytopathic effect was observed, leading to a decrease of luciferase signal at 48 h post-infection. The results suggest that luciferase signal could be used as a surrogate to test antiviral inhibitors.

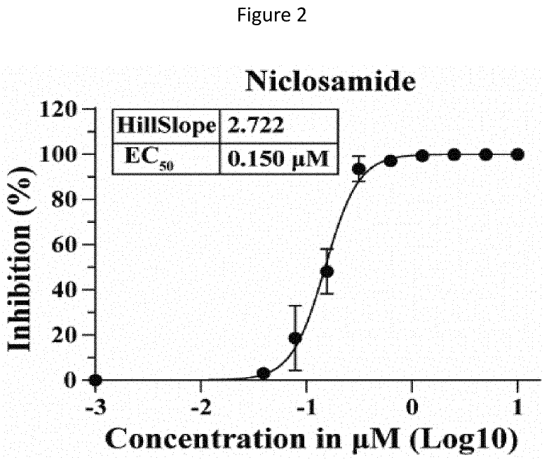

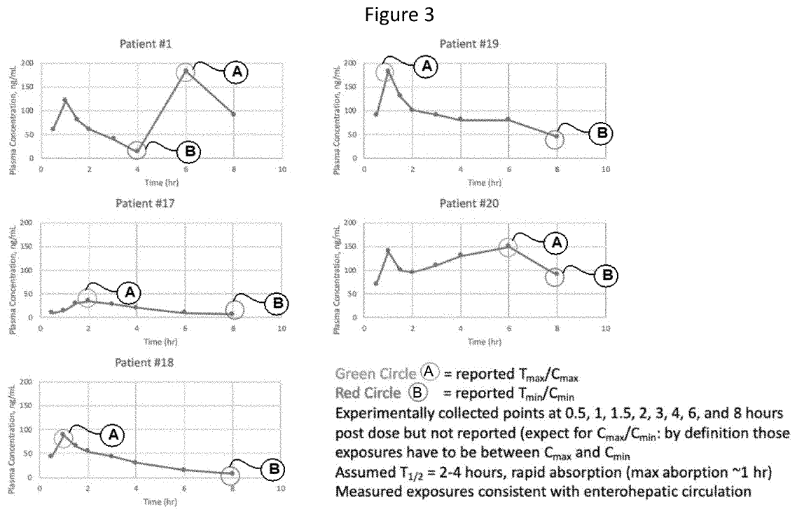

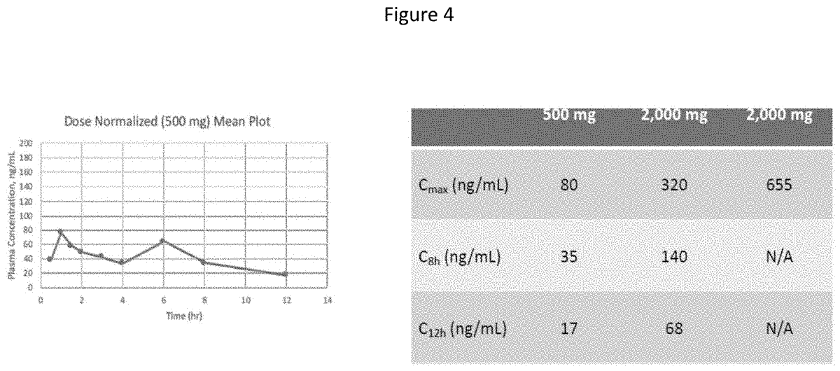

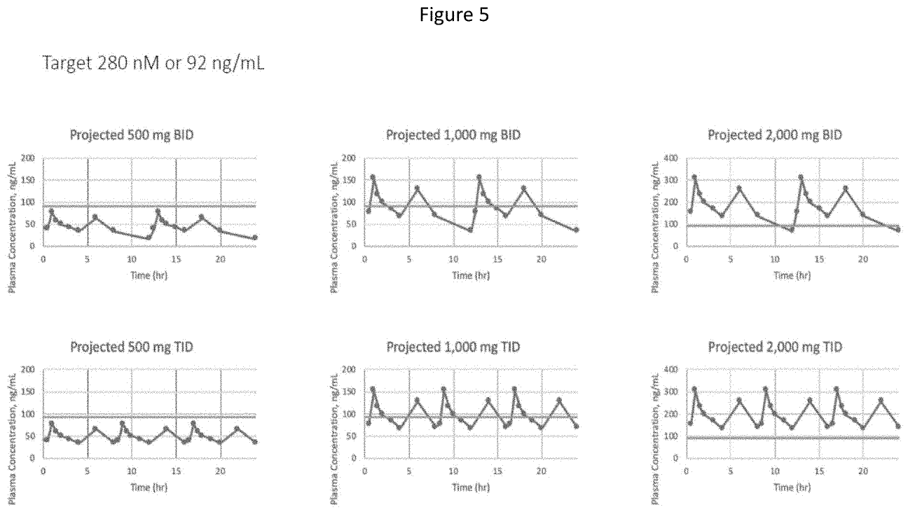

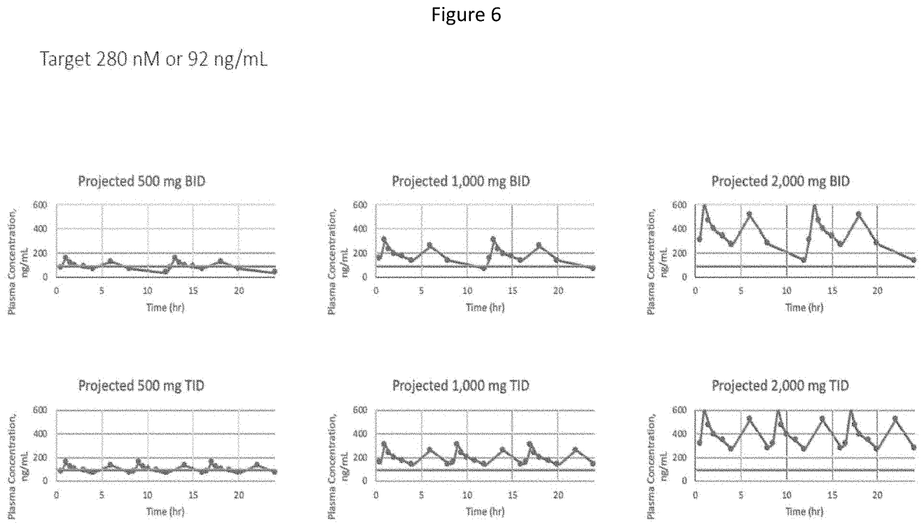

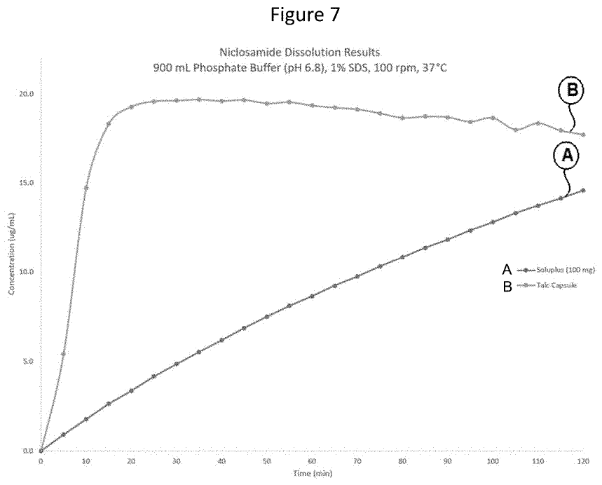

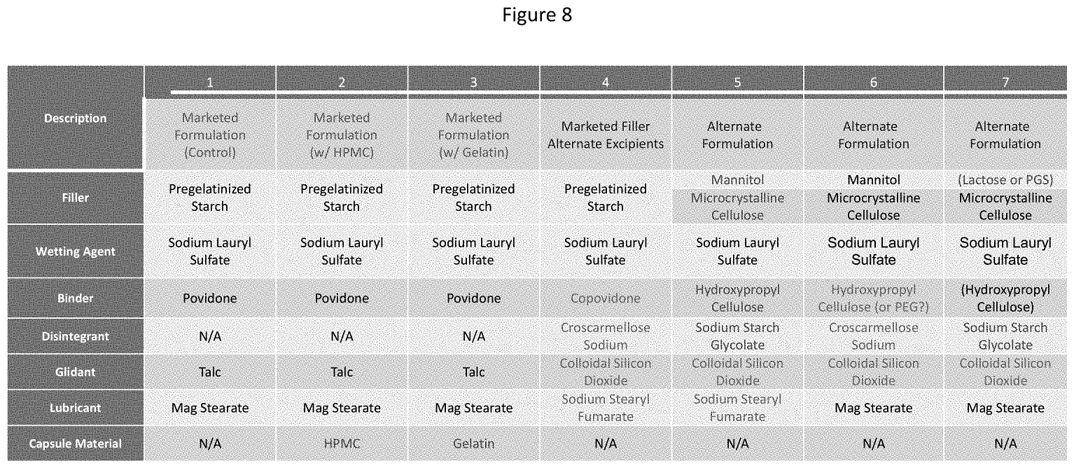

Niclosamide activity. Niclosamide is an antiparasitic agent that has broad spectrum of antiviral activity. To test its activity against SARS-CoV-2, niclosamide powder was dissolved in 100% DMSO at 10 mM. The antiviral assay was performed in a 96-well plate. Approximately 1.5×10⁴ Vero E6 cells were seeded to each well the night before viral infection. Different concentrations of niclosamide were added to cells together with reporter SARS-CoV-2 (MOI of 0.1). At each tested condition, the cell medium contained a final concentration of 0.5% DMSO. After 24 h post-infection, the infected cells were assayed for luciferase activity using Nano-Glo Luciferase System following the manufacture's protocol (Promega). The EC₅₀ value was derived from the luciferase curve (FIG. 2). Niclosamide showed a potent antiviral EC₅₀ of 150 nM.

### Example 2

In vitro, niclosamide is 10× more active than remdesivir against SARS-CoV-2. The disclosure provides dissolution studies of two formulations of niclosamide to determine the release profile of two niclosamide formulations.

Niclosamide (28.7 mg) was dissolved in EtOH (15 mL). A portion of this solution (0.2 mL) was diluted to 10 mL with EtOH to produce a niclosamide standard solution containing 38.3 μg/mL.

Monobasic sodium phosphate monohydrate (13.8 g) and SDS (20 g) were added to about 1,700 mL of water. Sodium hydroxide solution (0.2 N, 144 mL) was added then the mixture was stirred until homogeneous. The pH of the solution was adjusted to 6.8 using the NaOH solution. Nine hundred milliliters of the buffer solution was transferred to a well of a Van Kel VK7000 dissolution apparatus. The media was stirred at 100 rpm and was brought to 37° C.

One niclosamide capsule (i.e., a talc formulation) was added to the well and its release was monitored using a SI Photonics submergible dip probe (0.5 cm path length, full-spectrum (200 nm-450 nm) taken every 5 minutes over 2 h). The spectral data was exported in Excel. The UV intensity values at 440 nm were subtracted from the values at 341 nm to account for baseline drift. The resulting UV intensity values at each time point were converted to concentration (ug/mL) released using the results from the niclosamide standard then results were plotted vs time.

The Soluplus (excipient produced by BASF) niclosamide sample (102.2 mg) was analyzed in the same way. Additional information about Soluplus available at (pharmaceutical.basf.com/global/en/drug-formulation/products/soluplus.html).

The results of this comparative study show that the talc formulation has better release properties than the Soluplus formulation.

### Example 3

### Single Increasing Dose Study

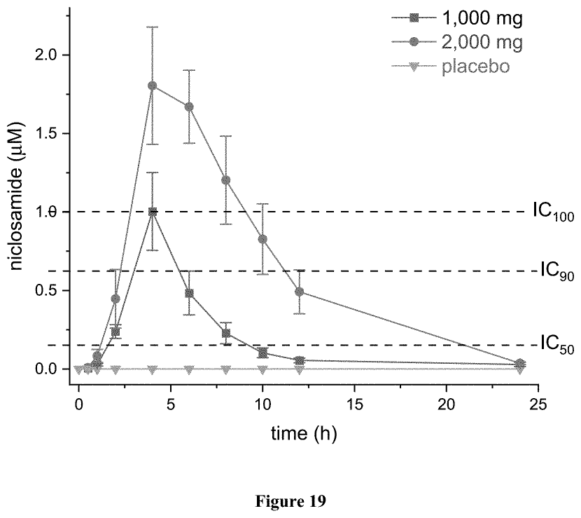

Healthy human subjects were randomized in a 4:1 ratio to receive a single oral dose of either final formulation (1,000 mg or 2,000 mg) or matching placebo. Blood for PK analysis was collected at timepoints t=0, 0.5, 1, 2, 4, 6, 8, 10, 12, 24 hours post-dose. FIG. 19 shows the concentration of niclosamide in human plasma at the respective timepoints. As shown in the figure, the concentration of niclosamide exceeded the respective in vitro IC₅₀, IC₉₀ and IC₁₀₀ values against SARS-CoV-2 for several hours after dosing.

### Example 4

### Multiple Increasing Dose Study

Healthy human subjects are randomized in a 3:1 ratio to receive oral doses of either final formulation (1,000 mg) or placebo on a twice per day (BID) schedule for seven consecutive days. Blood for PK analysis is collected at timepoints t=0, 0.5, 1, 2, 4, 6, 8, 10, 12, 24 hours post-dose. Blood samples will also be collected on Days 2, 4, 6, and 8 at pre-dose and before the 12-hour dose on Days 2, 4, and 6. It is expected that the concentration of niclosamide will be above the IC₅₀ and the IC₉₀ values for an extended period of time.

### Example 5

### Comparison of Bioavailabilities of Table 1 Formulation to Chewable Niclosamide Tablets (Yomesan®) of Upon Oral Administration

Published results show that oral administration of a single dose of 2,000 mg of niclosamide reached maximal systemic serum concentrations (Cₘₐₓ) in humans of 0.76-18.3 μM (249-5,986 ng/mL) (See, Andrews et al., Pharmac. Ther. Vol. 19, 1983, p. 245-298). A study in prostate cancer patients showed that 149-182 ng/mL (0.46-0.56 μM) become available after a single oral dose of 1,000 mg (See, Schweizer et al., PLOS ONE, Jun. 1 2018, p. 1-12). In a recent study, colorectal cancer patients received 2,000 mg of niclosamide orally once a day until disease progression or toxicity was observed (up to four months). Plasma levels peaked 240 minutes (tₘₐₓ) after the first niclosamide administration in the majority of patients with a median Cₘₐₓ of 2.03 μM (665 ng/mL) (See, Burock et al., J. Clinical. Oncology, 2020, Vol. 36, No. 15 Suppl., 1-4).

As shown in FIG. 19, administration of 1,000 mg of the final formulation provided a Cₘₐₓ of 1.00 μM, which is 1.8-fold to 2.2-fold greater than what Schweizer reported. Administration of 2,000 mg niclosamide, provided a Cₘₐₓ of 590 ng/mL (1.80 μM), and a tₘₐₓ of 4 h. This Cₘₐₓ value exceeds the lower bound of the values reported by Andrews by 2.4-fold, demonstrating the improved bioavailability of the claimed formulation.

1-277. (canceled)

278. A formulation comprising niclosamide in a dry and solid form, Sodium Lauryl Sulfate (SLS), and one or more of a surfactant, an excipient, a pH modulator, a preservative, a binder, a glidant, a dispersant, a disintegrant, and a filler.

279. The formulation of claim 278, wherein the SLS comprises at least 3% by weight of the formulation.

280. The formulation of claim 278, wherein the SLS comprises between about 3% and about 7% weight by weight of the formulation or between about 4% and about 6% weight by weight of the formulation.

281. A formulation comprising
(a) niclosamide, comprising about 55% to about 56% weight by weight of the formulation;
(b) mannitol, comprising about 8% to about 9% weight by weight of the formulation;
(c) Microcrystalline cellulose (MCC), comprising about 15% to about 16% weight by weight of the formulation;
(d) SLS, comprising about 5% weight by weight of the formulation;
(e) Sodium Starch Glycolate, comprising about 8% weight by weight of the formulation;
(f) Hydroxypropyl cellulose (HPC), comprising about 5% weight by weight of the formulation;
(g) Colloidal Silicon Dioxide, comprising about 0.5% weight by weight of the formulation; and
(h) Sodium Stearyl Fumarate, comprising about 2% weight by weight of the formulation.

282. The formulation of claim 281, comprising
(a) niclosamide, comprising about 55.5% weight by weight of the formulation;
(b) mannitol, comprising about 8.8% weight by weight of the formulation;
(c) MCC, comprising about 15.1% weight by weight of the formulation;
(d) SLS, comprising about 5% weight by weight of the formulation;
(e) Sodium Starch Glycolate, comprising about 8% weight by weight of the formulation;
(f) (HPC), comprising about 5% weight by weight of the formulation;
(g) Colloidal Silicon Dioxide, comprising about 0.5% weight by weight of the formulation; and
(h) Sodium Stearyl Fumarate, comprising about 2% weight by weight of the formulation.

283. The formulation of claim 281, comprising
(a) about 250 mg of niclosamide;
(b) about 39.6 mg of mannitol,
(c) about 68.1 mg of MCC;
(d) about 22.5 mg of SLS;
(e) about 36 mg of Sodium Starch Glycolate;
(f) about 22.5 mg of (HPC);
(g) about 2.3 mg of Colloidal Silicon Dioxide; and
(h) about 9 mg of Sodium Stearyl Fumarate.

284. A formulation comprising (i) intra-granular components and (ii) extra-granular components;
wherein the intra-granular components comprise:
(a) niclosamide, comprising about 55% to about 56% weight by weight of the formulation;
(b) mannitol, comprising about 8% to about 9% weight by weight of the formulation;
(c) MCC, comprising about 15% to about 16% weight by weight of the formulation;
(d) SLS, comprising about 5% weight by weight of the formulation;
(e) Sodium Starch Glycolate, comprising about 5% weight by weight of the formulation;
(f) (HPC), comprising about 5% weight by weight of the formulation;
(g) Colloidal Silicon Dioxide, comprising about 0.3% weight by weight of the formulation; and
(h) Sodium Stearyl Fumarate, comprising about 1% weight by weight of the formulation; and
wherein the extra-granular components comprise:
(i) Sodium Starch Glycolate, comprising about 3% weight by weight of the formulation;
(j) Colloidal Silicon Dioxide, comprising about 0.2% weight by weight of the formulation; and
(k) Sodium Stearyl Fumarate, comprising about 1% weight by weight of the formulation.

285. The formulation of claim 284, comprising (i) intra-granular components and (ii) extra-granular components;
wherein the intra-granular components comprise:
(a) niclosamide, comprising about 55.6% weight by weight of the formulation;
(b) mannitol, comprising about 8.8% weight by weight of the formulation;
(c) MCC, comprising about 15.1% weight by weight of the formulation;
(d) SLS, comprising about 5% weight by weight of the formulation;
(e) Sodium Starch Glycolate, comprising about 5% weight by weight of the formulation;
(f) (HPC), comprising about 5% weight by weight of the formulation;
(g) Colloidal Silicon Dioxide, comprising about 0.3% weight by weight of the formulation; and
(h) Sodium Stearyl Fumarate, comprising about 1% weight by weight of the formulation; and
wherein the extra-granular components comprise:
(i) Sodium Starch Glycolate, comprising about 3% weight by weight of the formulation;
(j) Colloidal Silicon Dioxide, comprising about 0.2% weight by weight of the formulation; and
(k) Sodium Stearyl Fumarate, comprising about 1% weight by weight of the formulation.

286. The formulation of claim 284, comprising (i) intra-granular components and (ii) extra-granular components;
wherein the intra-granular components comprise:
(a) about 250 mg of niclosamide;
(b) about 39.6 mg of mannitol;
(c) about 68.1 mg of MCC;
(d) about 22.5 mg of SLS;
(e) about 22.5 mg of Sodium Starch Glycolate;
(f) about 22.5 mg of (HPC);
(g) about 1.4 mg of Colloidal Silicon Dioxide; and
(h) about 4.5 mg of Sodium Stearyl Fumarate; and
wherein the extra-granular components comprise:
(i) about 13.5 mg of Sodium Starch Glycolate;
(j) about 0.9 mg of Colloidal Silicon Dioxide; and
(k) about 4.5. mg of Sodium Stearyl Fumarate.

287. The formulation of any one of claim 278, wherein the formulation shows higher percentage of drug release than a reference formulation, a Cₘₐₓ after oral dosing that is between about 1.5 and 2.5 times higher than a reference formulation, and/or higher systemic bioavailability than a reference formulation, wherein the reference formulation comprises 500 mg niclosamide, maize starch, talcum, sodium lauryl sulphate, povidone, vanillin, magnesium stearate, and saccharin sodium.

288. A method for treating or preventing a viral infection, comprising administering an effective amount of the formulation of claim 278 to a subject, wherein, upon contacting an infectious agent, the formulation prevents transmission, infection, replication, survival or growth of the virus in the subject.

289. The method of claim 288, wherein the virus belongs to the family of Picornaviridae, Astroviridae, Caliciviridae, Hepeviridae Flaviviridae, Togaviridae, Arteriviridae, or Coronaviridae.

290. The method of claim 289, wherein the virus belongs to the family of Coronaviridae.

291. The method of claim 290, wherein the Coronaviridae virus is Bat coronavirus CDPHE15, Bat coronavirus HKU10, Rhinolophus ferrumequinum alphacoronavirus HuB-2013, Human coronavirus 229E, Lucheng Rn rat coronavirus, Ferret coronavirus, Mink coronavirus 1, Miniopterus bat coronavirus 1, Miniopterus bat coronavirus HKU8, Myotis ricketti alphacoronavirus Sax-2011, Nyctalus velutinus alphacoronavirus SC-2013, Porcine epidemic diarrhea virus, Scotophilus bat coronavirus 512, Rhinolophus bat coronavirus HKU2, Human coronavirus NL63, NL63-related bat coronavirus strain BtKYNL63-9b, Human coronavirus OC43, China Rattus coronavirus HKU24, Human coronavirus HKU1, Murine coronavirus-type species, Bat Hp-betacoronavirus Zhejiang2013, Hedgehog coronavirus 1, Middle East respiratory syndrome-related coronavirus (MERS-CoV), Pipistrellus bat coronavirus HKU5, Tylonycteris bat coronavirus HKU4, Rousettus bat coronavirus GCCDC1, Rousettus bat coronavirus HKU9, Severe acute respiratory syndrome-related coronavirus, Wigeon coronavirus HKU20, Bulbul coronavirus HKU11-type species, Porcine coronavirus HKU15, Munia coronavirus HKU13, White-eye coronavirus HKU16, Night heron coronavirus HKU19, Common moorhen coronavirus HKU21, Beluga whale coronavirus SW1 or Avian coronavirus-type species.

292. The method of claim 291, wherein the Coronaviridae virus is Middle East respiratory syndrome-related coronavirus (MERS-CoV), Severe acute respiratory syndrome coronavirus (SARS-CoV) or Severe acute respiratory syndrome coronavirus 2 (SARS-CoV-2).

293. The method of claim 288, wherein the subject has a medical condition that increases risk of a poor prognosis without treatment or an increased risk of a severe immune response to the infection.

294. The method of claim 293, wherein the subject has an impaired or reduced immune response.

295. The method of claim 294, wherein the subject is immunocompromised.

296. The method of claim 288, wherein the subject is undergoing or has undergone one or more of an immunosuppressive therapy, presenting a sign or symptom of a second medical disease or disorder or a subject of a past, present or future medical intervention.

297. A method for preparing the formulation of claim 278, the method comprising combining niclosamide, mannitol, MCC, SLS, Sodium Starch Glycolate, HPC, Colloidal Silicone Dioxide, and Sodium Stearyl Fumarate.
